(19) United States
(12) Patent Application Publication
(10) Pub. No.: US 2003/0032203 A1
(43) Pub. Date: Feb. 13, 2003

(54) Small molecule microarrays

(76) Inventors: David M. Sabatini, Cambridge, MA (US); Brent R. Stockwell, Boston, MA (US)

Correspondence Address: ROPES & GRAY, ONE INTERNATIONAL PLACE, BOSTON, MA, 02110-2624, US

(21) Appl. No.: 10/189,336

(22) Filed: Jul. 10, 2002

Related U.S. Application Data
(60) Provisional application No. 60/304,253, filed on Jul. 10, 2001.

(51) Int. Cl. G01N 33/543

(52) U.S. Cl. 436/518

(57) ABSTRACT

Small molecule arrays, particularly small molecule microarrays, and methods of identifying a small molecule based on observing the effect of a small molecule on an observable characteristic of a biological sample or test element, such as a cell, protein, cell lysate, tissue slice or small organism.

### BACKGROUND OF THE INVENTION

Enhancing the traditional paradigm of small molecule discovery, combinatorial chemistry has resulted in a dramatic increase in the number of compounds that are available for screening, and human genome research has uncovered large numbers of new molecular targets for screening. A major goal of biomedical research is the identification of molecules and compounds that can modulate specific biological processes. Screens may use these new targets in a variety of ways, searching for enzyme inhibitors, receptor agonists or antagonists. The traditional goal is to find compounds that reduce, block, or enhance a single crucial interaction in a biological system (Weber et al., (1995) Angew. Chem. Int. Ed. Engl., 34:2280). The development of high-throughput assays to screen large collections of molecules and identify those that can interact with a specific protein target has been a major goal of academic and industrial research laboratories. However, the majority of assays employed in these screens either detect specific protein-ligand interactions using recombinant proteins or study the effects of small molecules on the growth of cells, without concern for the specific signaling pathways involved (Borchardt et al., supra; Huang & Schreiber. (1997) PNAS 94:13396; Combs et al. (1996) J. Am. Chem. Soc. 118:287).

Additionally, a number of researchers are adapting phenotype-based assay systems, where the screening is performed on whole, living cells, and the readout of the screen is some detectable property of the cell (Stockwell et al. (1999) Chem. Biol. 6:71; Mayer et al. (1999) Science 286:971).

Appropriately designed cell-based assays have the potential to identify small molecules that affect specific signaling pathways in vivo. There remains a need for the development of high throughput formats for screening compounds that can participate in or disrupt biological processes. There is a particular need for the development of high throughput systems that allow the analysis of events occurring inside cells.

### SUMMARY OF THE INVENTION

Described herein are reagents and methods for identifying a small molecule (also referred to as “test compound” herein), combination of small molecules, and/or small molecule concentrations that produce at least one observable characteristic of a biological sample or test element, such as when contacted with tissue, cells, proteins, cell lysate, or small organisms. In one embodiment, the subject method can be used for identifying a small molecule(s) that produce a change in phenotypic characteristic (observable property) of cells. Further embodiments are directed to a method of identifying the effect(s) of small molecules on an observable characteristic of such other biological systems as proteins, cell lysates, tissue slices and small organisms.

In exemplary embodiments, the method is carried out on an array, preferably a microarray, which comprises a surface having test compounds spotted thereon in discrete defined (separate) locations. Optionally, the surface is porous or penetrable (such as in the case of a gel or fibrous matrix), and the test compounds are optionally spotted within the surface. In certain preferred embodiments, the subject array is a spatially addressable array of compounds. In such embodiments, the identity of the small molecule or small molecules, or small molecule concentration if such is being varied in the array, is known by its location in the array. One or more biological samples are contacted with the array in such a manner and under conditions appropriate for the small molecule entities of the array may interact with it. In the case of cells and tissues, the conditions can be selected so that small molecule entities may interact with extracellular and/or intracellular components of the cells. For example, a sample of cells is placed on the array in such a manner that when the small molecule(s) is released from the locations on the array, it makes contact with the cells, and either remains outside the cell (e.g., in contact with the cell membrane) and/or enters the cells, such as by an endocytic pathway or diffusion across the membrane. Optionally, when the surface is porous or penetrable, cells may be implanted within the surface. Small molecules to be assessed for their effects on an observable characteristic of a biological sample are referred to herein as test small molecules.

In certain embodiments of the subject small molecule arrays the test small molecules are affixed to a surface in discrete, defined spatial locations in high density (a large number of test small molecules per unit area) and in such a manner that the small molecule(s) are released from/diffuse from the surface and contact cells cultured on the discrete, defined locations.

One embodiment of the method allows assessment of a large number of test small molecules in a microarray format. In phenotype-based screening methods of this invention, a test compound is contacted with cells or tissues plated on the array's surface, such as in a microarray format, and its effect(s) on an observable characteristic(s) of the cell(s) determined, e.g., changes in phenotype. The phenotype which is monitored can be any observable characteristic of cells, such as those listed herein. A wide variety of cell types can be used in the present invention in order to screen test small molecules.

In one embodiment, the small molecule microarray of the present invention comprises a surface having affixed thereto test small molecule-polymer mixtures (e.g., one or more test small molecule(s) in a polymer) in discrete, defined locations and in high density. Test small molecules in such discrete, defined locations are released and come into contact with vicinal cultured cells; test small molecules become membrane-bound and/or enter into cells and/or contact a component made by the cells. In a particular embodiment, the small molecule microarrays of the present invention comprise a surface having affixed thereto, in discrete, defined locations and in high density, small molecule-polymer mixtures (e.g., small molecules encapsulated in or bound to a polymer), from which the small molecule being assessed is released. The small molecule enters and/or makes contact with vicinal cells and the resulting effect(s), if any, on cellular phenotype are determined.

Test small molecules can be spotted on a surface by means of a semi-permeable polymer or other matrix which acts as a barrier to immediate release, and include hydrogels and biodegradable polymers. The test compounds can be admixed with the matrix and spotted, or can be provided as one or more layers of a multi-layered spot and encapsulated (surrounded or sandwiched) by the matrix. Whichever embodiment, the material used to form the matrix is selected to adhere to the substrate on which the test compounds are to be arrayed. In preferred embodiments, the matrix/compound spots adhere to the substrate such that at least 75 percent of the arrayed spots remain adherent in the presence of cell culture media after 24 hours at up to 36° C., and even more preferably at least 85, 95 or even 99 percent of the arrayed spots remain adherent.

A wide variety of materials can be used to form the matrix, provided that the test small molecule-matrix can be affixed to the microarray surface in discrete, defined locations in high density and remain affixed thereto when cells are cultured thereon; are not toxic to the cells being cultured; and permit release of test small molecules so that they can make contact with and/or enter vicinal cells or their components. The matrix can be selected to result in release of test small molecules at a rate appropriate for the phenotypic characteristic(s) being observed, the type of cell being studied or the molecular weight or other physical properties of the test small molecules being assessed (such as hydrophobicity, polarity, etc). For example, the matrix can be selected to result in release of test small molecules over a wide range of time periods (e.g., a few minutes, one half to one hour, several to many hours, weeks, months or a year or more).

In a specific embodiment, methacrylate-based polymers are used to produce small molecule-methacrylate-based polymer microarrays. In other embodiments, the arrays are formed using poly(α-hydroxy acid) polymers such as polylactic acids, polyglycolic acids and mixtures and derivatives thereof.

In certain embodiments where the process of printing the array involves pin devices or other methods in which solvent volatility may be an issue, the matrix can be dissolved in a solvent system that has a low-volatility organic content. In certain preferred embodiments, at least 50 percent (v/v) of the solvent used to dissolve the matrix for printing has a vapor pressure of less than water, and even more preferably at least about 75, 90 percent or even 95 percent. Preferably, at least 50 percent of the solvent used as a vapor pressure less than 10 mm Hg, and even more preferably at least about 75, 90 percent or even 95 percent.

In certain embodiments, the matrix and test compound are dissolved in a polar, aprotic solvent in order to be prepared for printing the array on the matrix.

The matrix can contain further include one or more agents that promote cell adhesion to the spots or differentiation on the spots or other desirable cell characteristic on the spots. For example, the polymer spots can contain or be coated with such agents as poly-lysine, glycosaminoglycans, peptides such as RGD peptides, t-butylaminoethyl methacrylate or other molecules which promote attachment of the cells to the matrix spots. The polymer spots can also be treated in such a fashion as to promote differentiation of cells on the spots or other desirable cell characteristic. The array can also be coated with agents that, when exposed to environmental conditions, alter the spot surface—such as promote attachment of cells to the spots (e.g., exposure to an ion plasma formed from ambient air in a plasma cleaning chamber, commonly known as plasma cleaning).

The matrix can be derived from components, or contain agents, that modulate the rate of release of the test compounds, e.g., in a passive or inducible manner. Such as agents, merely to illustrate, can be hydrophobic or lipophilic, or render the release profile of the test compounds subject to an enviromnental cue such as pH, temperature or the presence of an enzyme or other molecule which promotes hydrolysis of the agent that regulates the release profile. In one embodiment, the matrix can be an electrically conductive polymer which promotes release of the test compounds when current is applied.

In certain preferred embodiments, where an inducible matrix system is used, it is an inducible biodegradable matrx.

The matrix can be provided on the substrate in the form of a spot or disk. While often small and affixed in high density, the spots must be of sufficient size for a sufficient number of cells to grow in diffusional proximity so as to permit contact of the test compounds and observation of phenotypic changes, if any, in the sample. For example, in certain embodiments, each spot covers a surface area of from about 0.0001 mm² to about 10 mm². In particular embodiments, each spot covers a surface area of from about 0.0001 mm² to about 0.001 mm²; from about 0.0001 mm² to about 0.01 mm^{2;} from about 0.0001 mm² to about 0.1 mm^{2 ;} from about 0.001 mm² to about 0.01 mm^{2;} or from about 0.005 mm² to about 0.01 mm^{2.} In one embodiment, each spot covers a surface area of approximately 0.01 mm². The spots can be a variety of shapes, e.g., circular, square, rectangular or other shape which permit discrete location.

In one embodiment, each spatial location or each row or column on an array comprises a different test compound, combination of test compounds and/or concentration of compounds, with the result that large numbers of small molecules can be tested or screened on a single microarray.

In certain embodiments, the small molecule microarray comprises a surface having affixed thereto test small molecule-biodegradable polymer mixtures in discrete, defined locations and preferably in high density. The biodegradable polymer in the mixture can be any biodegradable polymer that can retain a test small molecule at defined, discrete locations and from which the test small molecule is released in such a manner that it comes in contact with and/or enter into cells or their components cultured thereon. Cultured cell biodegrade the polymer, the test small molecule is released from the polymer and contacts and/or enters the cells or their components, and its effect(s), if any, on a phenotypic characteristic(s) of the cells are determined. A wide variety of biodegradable polymers can be used in this embodiment; including, but not limited to, poly(lactic acid), poly(glycolic acid), polylactide coglycolide, and gelatin. (See, for example, Biodegradable Hydrogels for Drug Delivery, Kinam Park et.al., Technomic Publishing Co., Inc, (1993). Such arrays are referred to herein as biodegradable polymer-based arrays; microarrays of this type are referred to as biodegradable polymer-based microarrays.

In the embodiments of the present invention, individual test tubes are not required, as is evident from the description that follows. However, for certain embodiments, such as for use with non-adherent cells cultures or protein solutions, wells or other depressions in the substrate may be advantageously used. The use of microfluidic channels and wells are contemplated for use in certain embodiments.

The test compound-matrix mixtures can be applied to low volume nanowells (wells of less than or equal to 1 microliter (μI). For example, in this method, each compound can be mixed with a matrix forming solution, e.g., for a biodegradable polymer and deposited in the bottom of a nanowell using a pin device such as a microarray spotter. After drying or other method of curing the polymer, cells can be added to the nanowells, (e.g., to all simultaneously or to individual wells) and excess medium removed. The matrix enables slow (e.g., greater than 10% of test small molecule released over 0.1 to 100 hours, such as 1 to 10 hours) release of test small molecule after cell addition.

Alternatively, a patterned surface containing alternating regions of hydrophobic and hydrophilic surfaces that permit cell growth only on the hydrophilic surfaces can be used. In this method, each test small molecule is mixed with a matrix and deposited on the hydrophilic surface. After drying or other method of curing, cells can be added to the surface (e.g., to the entire surface simultaneously) and excess medium is removed so that the hydrophobic surfaces are cleared of cells and medium. The patterned surface enables small droplets to form on each hydrophilic spot. A cover slip placed over droplets can prevent evaporation. The alternating hydrophobic/hydrophilic surfaces cause small aqueous droplets to form by surface tension only on the hydrophilic areas and water is excluded from the hydrophobic regions. This creates small volume wells, with each droplet forming its own well.

The microarrays of the present invention make it possible to test a large number of small molecules (e.g., as many as a quarter million or more test small molecules) in a single array which is the size of a standard microplate. It is reasonable to expect that this technology can be used to screen 10 million test small molecules per day in duplicate or, alternatively, 1 million test small molecules per day in 10 different cell-based assays in duplicate. Each compound (location containing a test small molecule) requires only a few cells for testing. For example, 5 to 10 cells can be used and as few as one, two, three or four cells can be used, making the present invention useful for screening small molecules in rare primary cells (primary cells available in limited number).

### BRIEF DESCRIPTION OF THE DRAWINGS

FIG. 1 is a schematic representation of conventional high-throughput small molecule screening (left panel) and of geldisk-based high-throughput small molecule screening.

FIG. 2 shows live cancer cells growing on a geldisk lacking toxic small molecules (1000X magnification).

FIG. 3 shows dead cancer cells on a geldisk containing a toxic small molecule (100 magnification) (phenylarsine oxide).

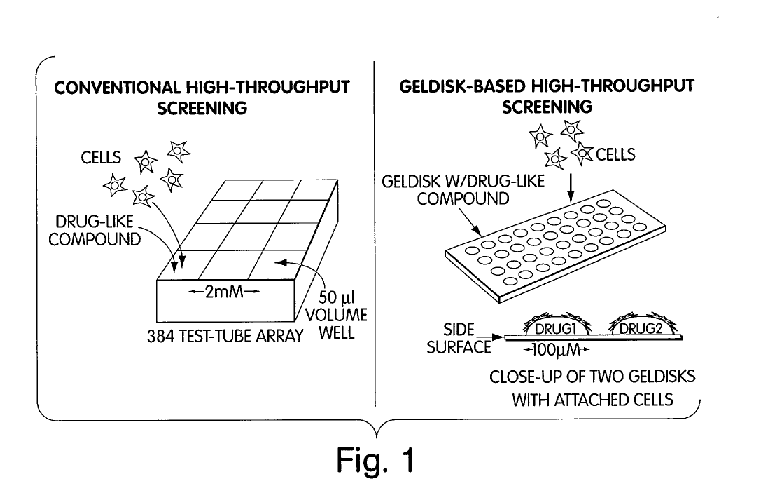

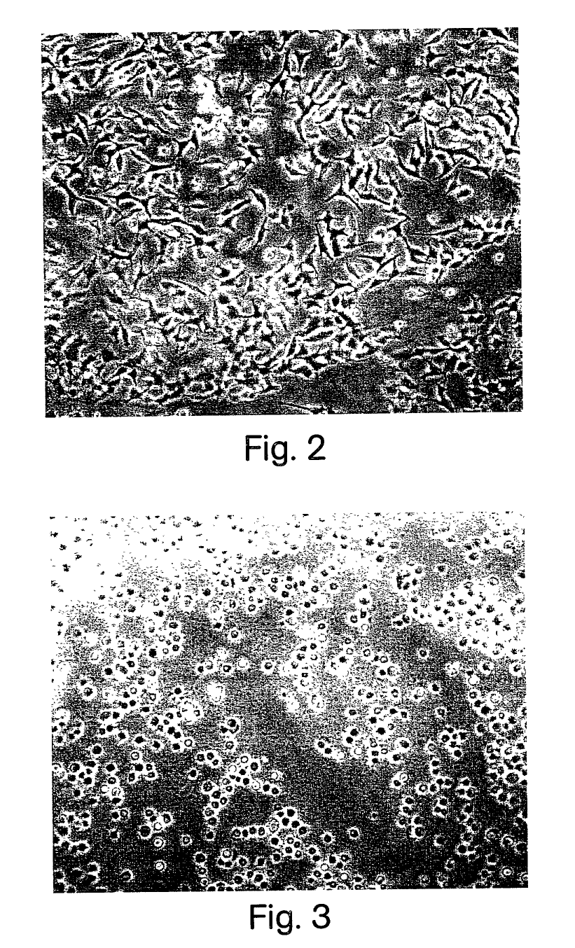

### DETAILED DESCRIPTION OF THE INVENTION

I. Overview

Described herein is a small molecule array, such as a small molecule microarray, that comprises a surface (e.g., a slide or other surface) having affixed thereto, in discrete, defined (separate) locations, at least one test compound to be-assessed for their effect(s) on a biological sample or test element, such as cell(s), protein(s), cell lysate, tissue slice or small organism, such as worms (e.g., Caenorhabditis (C.) elegans), flies (e.g., Drosophila (D.) melanogaster), yeast (e.g., Saccharomyces (S.) cerevisiae or Zebrafish (e.g., Danio (D.) rerio). Test compounds that can be screened to determine whether they alter or modify cell phenotype, using a small molecule microarray of this invention, include proteins, peptides, polynucleotides (DNA,RNA), small organic molecules and other compounds or molecules that do not need to be expressed in cells. Once a test small molecule has been shown to be a small molecule (e.g., has been shown to alter/modify a characteristic of the biological sample, such as cell phenotype), it can be further assessed, using in vitro and/or in vivo methods, to confirm the observed effect(s) and, optionally, to determine the mechanism by which it acts on the characteristic, such as cell phenotype. For example, known methods such as transcription profiling affinity chromatography, transfected cell assays and assessments in appropriate animal models, can be used to identify/detect changes in phenotypic characteristics.

II. Definitions

By “array” is meant a multi-dimensional arrangement, preferrably two dimensional, of test compounds whereby the identity of a compound or combination of compounds, or concentration of compound(s), can be identified by its spatial location in the array.

By “test compounds” is meant a number of chemical compounds which are to be screened for ability to effect physiological parameters of a cell or tissue. In certain embodiments, the test compounds are chemical compounds, e.g., small organic molecules, generated by conventional or combinatorial chemistry methods. In other embodiments, the test compounds are chemical compounds which are naturally occurring compounds more or less purified from their native state, e.g., natural extracts.

A “polar solvent” means a solvent which has a dielectric constant (ε) of 2.9 or greater, such as DMF, THF, ethylene glycol dimethyl ether (DME), DMSO, acetone, acetonitrile, methanol, ethanol, isopropanol, n-propanol, t-butanol or 2-methoxyethyl ether. Preferred solvents are DMF, DME, NMP, and acetonitrile.

An “aprotic solvent” means a non-proton containing solvent having a boiling point range above ambient temperature, preferably from about 25° C. to about 190° C., more preferably from about 80° C. to about 160° C., most preferably from about 80° C. to 150° C., at atmospheric pressure. Examples of such solvents are acetonitrile, toluene, DMF, diglyme, THF or DMSO.

A “polar, aprotic solvent” means a polar solvent as defined above which has no available hydrogens to exchange with the compounds of this invention during reaction, for example DMF, acetonitrile, diglyme, DMSO, or THF.

The “vapor pressure” of a compound is defined as the pressure at any given temperature (standard T is 25° C.) of a vapor (gas) in equilibrium with its liquid. When the liquid is pure, the resulting pressure is called the saturation vapor pressure and given the symbol P∘.

The term “volatility rating” describes how quickly a liquid solvent will evaporate (how quickly it goes into the air as a vapor). The ratings are based on vapor pressure at 25° C.

Less than 1 mmHg: VERY LOW

1-10 mmHg:LOW

10-100 mmHg:MEDIUM

100-760 mmHg: HIGH

More than 760 mmHg: Gas at room temperature.

The term “biodegradable” with respect to a polymer or hydrogel means that the matrix of the polymer or gel loses dimensional stability over time when subjected to a biological environment, such as under cell culture conditions.

The term “biocompatible” as used herein with respect to a polymeric or hydrogel system means that neither the polymer or gel, nor its degradation products, are toxic or elicit an adverse biologic response in cultured cells or tissues.

The term “member” as used herein refers to one of a plurality of chemical compounds which together form a chemical library.

The term “feature”, as it is used in describing an array, refers to an area of a substrate having a homogenous collection of a test compound (or compounds in the case of certain combinatorial embodiments). One feature can be different than another feature if the test compounds of the different features have different structures or different concentrations.

As used herein, “signal” is the measured phenotype conferred on a target cell by a library member. Examples of signals may be, but are not limited to, fluorescence, fluorescence polarization, luminescence, radiation, absorption of radiation, electromotive potential, pH, enzyme activity, and cell growth, differentiation and/or death. The intensity of the signal may be directly or inversely proportional to some desirable property for which the library is being assayed.

The term “loss-of-function”, as it refers to the effect of a test compounds, refers to those test compounds that, when contacted with a target cell or tissue, inhibit expression of a gene or otherwise render the gene product thereof to have substantially reduced activity, or preferably no activity relative to one or more functions of the corresponding wild-type gene product.

As used herein, a “desired phenotype” refers to a particular phenotype for which the user of the subject method seeks to observe from the target cell or tissue upon contact with one or more test compounds.

As used herein, the term “nucleic acid” refers to polynucleotides such as deoxyribonucleic acid (DNA), and, where appropriate, ribonucleic acid (RNA).

As used herein, the terms “heterologous nucleic acid” and “foreign nucleic acid” refer to a nucleic acid, e.g., DNA or RNA, that does not occur naturally as part of the genome in which it is present or which is found in a location or locations in the genome that differs from that in which it occurs in nature. Heterologous DNA is not endogenous to the cell into which it is introduced, but has been obtained from another cell. Examples of heterologous nucleic acid include, but are not limited to, DNA that encodes test polypeptides, receptors, reporter genes, transcriptional and translational regulatory sequences, selectable or traceable marker proteins, such as a protein that confers small molecule resistance.

As used herein, “recombinant cells” include any cells that have been modified by the introduction of heterologous nucleic acid. Control cells include cells that are substantially identical to the recombinant cells, but do not express one or more of the proteins encoded by the heterologous nucleic acid.

The terms “protein”, “polypeptide” and “peptide” are used interchangeably herein.

The terms “recombinant protein”, “heterologous protein” and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

As used herein, “cell surface receptor” refers to molecules that occur on the surface of cells, interact with the extracellular environment, and transmit or transduce the information regarding the environment intracellularly in a manner that may modulate intracellular second messanger activities or transcription of specific promoters, resulting in transcription of specific genes.

As used herein, “extracellular signals” include a molecule or a change in the environment that is transduced intracellularly via cell surface proteins that interact, directly or indirectly, with the signal. An extracellular signal or effector molecule includes any compound or substance that in some manner alters the activity of a cell surface protein. Examples of such signals include, but are not limited to, molecules such as acetylcholine, growth factors and hormones, lipids, sugars and nucleotides that bind to cell surface and/or intracellular receptors and ion channels and modulate the activity of such receptors and channels. The term also include as yet unidentified substances that modulate the activity of a cellular receptor, and thereby influence intracellular functions. Such extracellular signals are potential pharmacological agents that may be used to treat specific diseases by modulating the activity of specific cell surface receptors.

“Orphan receptors” is a designation given to a receptors for which no specific natural ligand has been described and/or for which no function has been determined.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences is directly or indirectly regulated by the target receptor protein. Exemplary transcriptional control sequences are promoter sequences. A reporter gene is meant to include a promoter-reporter gene construct which is heterologously expressed in a cell.

“Signal transduction” is the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylate cyclase, direct activation (or inhibition) of a transcriptional factor and/or activation.

The term “modulation of a signal transduction activity of a receptor protein” in its various grammatical forms, as used herein, designates induction and/or potentiation, as well as inhibition of one or more signal transduction pathways downstream of a receptor.

The term “spot” as used in reference to a placement of test compound on or within the surface of the array is intended to include any geometry that permits placement of test compounds at discrete defined locations. A “spot” may have substantial depth, width and length. Exemplary spots include relatively flat, circular placements, roughly spherical placements (particularly where the test compound is inserted into a porous or permeable surface), stripes, columns, squares, cubes, etc.

III. Exemplary Matrix Systems

Test small molecules are affixed to the surface by means of a matrix, also referred to as a semi-permeable polymer that immobilized them to the surface (prevent immediate release from the surface); permits release of the small molecule at an appropriate rate under the conditions under which an assay is carried out; permits the cells used to attach and is not toxic to the cells.

In certain embodiments, the release rate of test compounds from the array can be determined principally by diffusion from the matrix. However, in certain preferred embodiments, the matrix is erodible, and the rate of erosion of the matrix is rate limiting for the rate of release of the test compounds.

In certain embodiments, the matrix is a biodegradable polymer. A number of suitable biodegradable polymers for use in making the arrays of this invention are known available and can be easily identified. Such polymers include natural polymers such as fibrin, hyaluronic acid, and collagen, as well as synthetic polymers such as polyanhydrides and aliphatic polyesters. The polymers can be used alone or in combination with other agents, e.g., other biomaterials typically used in tissue engineering.

Merely to illustrate, synthetic polymers that can be utilized for the instant applications include poly(α-hydroxy acids) [e.g., polylactic acid (PLA), polyglycolic acid (PGA), polycapronic acid], poly(orthoesters), polyurethanes and hydrogels [e.g.,polyhydroxyethylmethacrylate, polyglycidylmethacrylate/diethylene glycol dimethacrylate or polyethylene oxide/polypropyleneoxide copolymers].

In certain embodiments, the matrix is formed using one or more of polyethylene glycol (PEG); poly(ethylene terepthalate; Dacron™) fibers; polyanhydrides; polyamides; polyphosphazenes; tricalcium phosphate and hydroxyapatite ceramic. Polycarbonates, polyfumarates and caprolactones may also be used to make the arrays of this invention.

In certain preferred embodiments, the matrix is a biodegradable poly(α-hydroxyl acid), such as a biodegradable polylactic or polyglycolytic acid, or a co-polymer thereof. Homopolymers or copolymers of lactide or glycolide are non-toxic, biocompatible, and biodegradable. As described in the appended examples, such biodegradable polymers as PLA, PDLA, PDLGA had greater than 99 percent spot attachment after 24 hours to all surfaces tested.

Polylactic acid (PLA) and polyglycolic acid (PGA) are derivatives of cyclic diesters of lactic and glycolic acid from which they have been produced by ring opening polymerization, resulting in poly-α-hydroxy derivatives of the original acids. The polymers are composed of macromolecules with molecular weights typically from tens of thousands of daltons to more than 1 million daltons. Exemplary poly(α-hydroxy acids) include:

PDLA: poly-D-lactide

PDLLA: poly-DL-lactide (50:50)

PDS: polydioxanon

PGA: polyglycolic acid or polyglycolide

PLA: polylactic acid or polylactide

PLA96: poly-L,D-lactide (96% L-lactide, 4% D-lactide)

PLA 85/15: poly-L,D-lactide (85% L-lactide, 15% D-lactide)

P(L/DL)LA: poly-L,DL-lactide

P(L/DL)LA 70/30: poly-L,DL-lactide (70% L-lactide, 30% DL-lactide)

PLGA: copolymer of polylactide and polyglycolide

PDLGA: copolymer of DL-polylactide and polyglycolide

PLGA70/30: copolymer of polylactide and polyglycolide (70% polylactide, 30% polyglycolide)

PLLA: poly-L-lactide

In certain embodiments, the matrix of the invention are provided by a copolymer in the form of a biocompatible, biodegradable, copolymer comprising a first backbone molecule of PLA bonded via a cross-linking reaction to a second backbone molecule of dextran or PEG wherein the dextran provides multiple hydroxyl functionalities.

In certain embodiments, the matrix is a hydrogel, such a polycarboxylic acid, cellulosic polymer, polyvinylpyrrolidone, malcic anhydride polymer, polyamide, polyvinyl alcohol, or polyethylene oxide.

In certain other embodiments, the matrix is an alginate.

In certain embodiments, the matrix is a photopolymerizable matrix, and even more preferably is a photopolymerizable biodegradable hydrogel. Merely to illustrate, the matrix can be a biodegradable, photopolymerizable matrix formed from a substantially water soluble macromer comprising components P, B, and L, wherein P comprises an organic group capable of being crosslinked by photopolymerization, L is a linking group, comprising at least one repeating unit, and having at least one of the properties of water solubility or biodegradability, and B is a backbone group, comprising at least one repeating unit, and having at least one of the properties of water solubility or biodegradability.

Solvents utilizable in the present invention include both organic and inorganic solvents. A salient feature to certain embodiments of the polymer systems is that the polymer/solvent be sufficiently flowable (liquid) to be printed on the surface using a pin device or other means for delivering fluids in the nanoliter volume range. At the same time, the solvent should be selected such that the test compound are miscible in it, and it is not so volatile as to evaporate in the printing device (e.g., so as to clog the device). In certain instances, it must also be compatible with the growth of cells. Exemplary solvents for use in the present invention include hexanoic acid, heptanoic acid, octanoic acid, pelargonic acid, decanoic acid, neodecanoic acid, benzoic acid, salicylic acid, cinnamic acid, o-toluic acid, m-toluic acid, p-toluic acid, p-hydroxybenzoic acid, p-tert. butyl benzoic acid, azelaic acid, isophorone, methyl benzoate, ethyl benzoate, acetyl salicylic acid, adipic acid, sebacic acid, itaconic acid, malic acid, dodecanedioic acid, 2-benzoylbenzoic acid, benzyl benzoate, methyl salicylate, cyclohexanone, benzophenone, butyl ether, diethylene glycol dimethyl ether, anisole, diethylene glycol dibenzoate, 2-heptanone, 2-octanone, butyl benzoate, acetophenone, benzyl ether, diethylene glycol diethyl ether, adiponitrile, dipropylene glycol dibenzoate, ethylene glycol diacetate, glycerol triacetate, chloroform, acetonitrile, propionitrile, 1,4 dichlorobutane, triethylamine, ethylene dichloride, diethanolamine, bromoform, butyl acetate, 1-butanol, butyl methyl ketone, caprolactam, 1,2 dichloropropane, 1,4 dioxane, ethylene glycol, glycerol, 1,1,1,2-tetrachloroethane and glutaric acid.

In certain embodiments, the solvent system has a low-volatility organic content (less than 10 mm Hg) of at least 50 percent (v/v), preferably at least than about 75 percent, and more preferably at least 90 percent or even 95 percent. Preferably, the solvent system is less volatile than water. In certain preferred embodiments, the solvent system has a very low-volatility organic content (less than 1 mm Hg) of at least 50 percent (v/v), preferably at least than about 75 percent, and more preferably at least 90 percent or even 95 percent.

Exemplary solvents having a volatility rating of medium or less include dimethylacetamide (DMAC), dimethylformamide (DMF), dimethylsulfoxide (DMSO), ethylene glycol, propyleneglycol, 3-methyl(oxazolidinone), 1,3-dimethyl imidazolidinone (DMEN), propylene carbonate, ethylenecarbonate, methyl pyrrolidinone, diethanolamine, bromoform, 1-butanol, adiponitrile, caprolactam, glycerol and/or methyl salicylate. Where miscible, mixtures of any of the above may be used.

The solvent system may also include viscosity reducing solvents, such as acetonitrile, ethylacetate or tetrahydrofuran.

In certain embodiments, the organic solvent is essentially free of solvent components having a boiling point below about 30C, flash point below about 50C.

In certain embodiments, a matrix comprising test compound may be coated with or mixed with a material that facilitates adhesion of cells to the matrix (a “cell adhesive material”). For example, a matrix may be coated with fibronectin, collagen or polylysine. The concentration of the material to be applied as a coating may be optimized for the type of cell and degree of adhesion desired. In certain embodiments, a matrix, such as a matrix comprising glycidyl methacrylate, may be contacted with a solution of polylysine at a concentration ranging from 5-250 μg/ml, optionally a concentration ranging from 10-50 μg/ml and preferably about 15 μg/ml. In further embodiments, a matrix may be contacted with a solution of fibronectin at a concentration ranging from 20-200 μg/ml, optionally a concentration ranging from 40-80 μg/ml and preferably about 60 μg/ml.

IV Exemplary Substrates

Any suitable surface which can be used to affix the test compound containing matrices to its surface or within its surface can be used. For example, the surface can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), paper, biomaterials (such as protein, gelatin, agar), tissues (such as skin, endothelial tissue, bone, cartilage), minerals (such as hydroxylapatite, graphite). In certain embodiments, the surface is permeable or porous so as to permit placement of test compound within the surface. Examples of permeable or porous surfaces include hydrogels, many biomaterials; fibrous materials, etc. Additional compounds may be added to the base material of the surface to provide functionality. For example, scintillants can be added to a polystyrene substrate to allow Scintillation Proximity Assays to be performed. The substrate may be a porous solid support or non-porous solid support. The surface can have concave or convex regions, patterns of hydrophobic or hydrophilic regions, diffraction gratings, channels or other features. The scale of these features can range from millimeter to nanometer scale. For example, the scale can be on the micron scale for microfluidics channels or other MEMS features or on the nanometer scale for nanotubes or buckyballs. The surface can be planar, planar with raised or sunken features, spherical (e.g. optically encoded beads), fibers (e.g. fiber optic bundles), tubular (both interior or exterior), a 3-dimensional network (such as interlinking rods, tubes, spheres) or other shapes. The surface can be part of an integrated system. For instance, the surface can be the bottom of a microtitre dish, a culture dish, a culture chamber. Other components such as lenses, gratings, electrodes can be integrated with the surface. In general, the material of the substrate and geometry of the array will be selected based on criteria that it be useful for automation of array formation, culturing and/or detection of cellular phenotype.

In still other embodiments, the solid support is a microsphere (bead), especially a FACS sortable bead. Preferably, each bead is an individual feature, e.g., having a homogenous population of test compounds and distinct from most other beads in the mixture, and one or more tags which can be used to the identify any given bead and therefore the test compound it displays. The identity of any given test compound that can induce a FACS-detectable change in cells that adhere to the beads can be readily determined from the tag(s) associate with the bead. For example, the tag can be an electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the FACS sorting of the cell/bead entities and subsequent decoding by ECGC after oxidative detachment of the tag sets from isolated beads. In other embodiments, the beads can be tagged with two or more fluorescently active molecules, and the identity of the bead is defined by the ratio of the various fluorophores.

In still another embodiment, the test compound array can be disposed on the end of a fiber optic system, such as a fiber optic bundle. Each fiber optic bundle contains thousands to millions of individual fibers depending on the diameter of the bundle. Changes in the phenotype of cells applied to the test compound array can be detected spectrometrically by conductance or transmittance of light over the spatially defined optic bundle. An optical fiber is a clad plastic or glass tube wherein the cladding is of a lower index of refraction than the core of the tube. When a plurality of such tubes are combined, a fiber optic bundle is produced. The choice of materials for the fiber optic will depend at least in part on the wavelengthes at which the spectrometric analysis of the cells on the array is to be accomplished.

In addition, the surface can be coated with, for example, a cationic moiety. The cationic moiety can be any positively charged species capable of electrostatically binding to negatively charged cellular membranes. Preferred cationic moieties for use are polycations, such as polylysine (e.g., poly-L-lysine), polyarginine, polyornithine, spermine, basic proteins such as histones (Chen et al. (1994) FEBS Letters 338:167-169), avidin, protamines (see e.g., Wagner et al. (1990) PNAS 87: 3410-3414), modified albumin (i.e., N-acylurea albumin) (see e.g., Huckett et al. (1990) Chemical Pharmacology 40: 253-263) and polyamidoamine cascade polymers (see e.g., Haensler et al. (1993) Bioconjugate Chem. 4:372-379). Alternatively, the surface itself can be positively charged (such as gamma amino propyl silane or other alkyl silanes).

The surface can also be coated with molecules for additional functions. For instance, these molecules can be capture reagents such as antibodies, biotin, avidin, Ni-NTA to bind epitopes, avidin, biotinylted molecules, or 6-His tagged molecules. Alternatively, the molecules can be culture reagents such as extracellular matrix, fetal calf serum, collagen.

V. Cells

Suitable target cells for generating the subject assay include prokaryotes, yeast, or higher eukaryotic cells, including plant and animal cells, especially mammalian cells. Prokaryotes include gram negative or gram positive organisms.

In certain preferred embodiments, the subject method is carried out using cells derived from higher eukaryotes, e.g., metazoans, and in especially preferred embodiments, are mammalian cells, and even more preferably are primate cells such as human cells. Other preferred species of mammalian cells include canine, feline, bovine, porcine, mouse and rat. For instance, such cells can be hematopoietic cells, neuronal cells, pancreatic cells, hepatic cells, chondrocytes, osteocytes, or myocytes. The cells can be fully differentiated cells or progenitor/stem cells.

Moreover, the cells can be derived from normal or diseased tissue, from differentiated or undifferentiated cells, from embryonic or adult tissue.

The cells may be dispersed in culture, or can be tissues samples containing multiple cells which retain some of the microarchitecture of the organ.

The choice of appropriate host cell will also be influenced by the choice of detection signal. For instance, reporter constructs can provide a selectable or screenable trait upon gain-of-function or loss-of-function induced by a test compound. The reporter gene may be an unmodified gene already in the host cell pathway, or it may be a heterologous gene (e.g., a “reporter gene construct”). In other embodiments, second messenger generation can be measured directly in a detection step, such as mobilization of intracellular calcium or phospholipid metabolism, in which case the host cell should have an appropriate starting phenotype for activation of such pathways.

In certain embodiments, the host cells are plated (placed) onto the surface bearing the test compound array in sufficient density and under appropriate conditions for introduction/entry of the test compounds into the cells. Preferably, the host cells (in an appropriate medium) are plated on the array at high density (e.g., on the order of 0.5-1×10⁵/cm²). For example, the density of cells can be from about 0.3×10⁵/cm² to about 3×10⁵/cm^{2 ,} and in specific embodiments, is from about 0.5×10⁵/cm² to about 2×10⁵/cm² and from about 0.5×10⁵/cm² to about 1×10⁵/cm². Optionally, where the surface of the array permits, cells may be implanted in the array.

In certain embodiments, the host cells can engineered to express recombinant genes. For instance, the host cells can be engineered with a reporter gene construct, and the ability of members of the test compound array to alter the level of expression of the reporter gene can be assessed. Merely to illustrate, the test compound array can be assessed for members which can function as transcriptional activators or transcriptional repressors of the reporter gene.

In other instances, the host cells can be engineered so as to have a loss-of-function or gain-of-function phenotype, and the ability of the ability of members of the test compound array to counteract such a phenotype is assessed.

In still other instances, the host cells are engineered to express a recombinant cell surface receptor, and the ability of one or more members of the library to induce or inhibit signal transduction by the receptor is assessed.

VI. Detection of Small molecule Activity

A variety of methods can be used to detect the consequence of uptake of the test compounds. In a general sense, the assay provides the means for determining if the test compound is able to confer a change in the phenotype of the cell relative to the same cell but which has not been contacted with the test compound. Such changes can be detected on a gross cellular level, such as by changes in cell morphology (membrane ruffling, rate of mitosis, rate of cell death, mechanism of cell death, dye uptake, and the like). In other embodiments, the changes to the cell's phenotype, if any, are detected by more focused means, such as the detection of the level of a particular protein (such as a selectable or detectable marker), or level of mRNA or second messenger, to name but a few. Changes in the cell's phenotype can be determined by assaying reporter genes (β-galactosidase, green fluorescent protein, β-lactamase, luciferase, chloramphenicol acetyl transferase), assaying enzymes, using immunoassays, staining with dyes (e.g. DAPI, calcofluor), assaying electrical changes, characterizing changes in cell shape, examining changes in protein conformation, and counting cell number. Other changes of interest could be detected by methods such as chemical assays, light microscopy, scanning electron microscopy, transmission electron microscopy, atomic force microscopy, confocal microscopy, image reconstruction microscopy, scanners, autoradiography, light scattering, light absorbance, NMR, PET, patch clamping, calorimetry, mass spectrometry, surface plasmon resonance, time resolved fluorescence.

For example, immunofluorescence can be used to detect a change in protein levels as a consequence to small molecule activity. Alternatively, small molecules that alter the phosphorylation state or subcellular localization of proteins, or that bind with proteins or with nucleic acids or proteins with enzymatic activity can be detected.

In one embodiment, the screen can be for the inability to grow or survive when a parasite or infectious agent is added to the cell of interest. In this case the selection would be for small molecules that inhibit targets that are specifically essential for some aspect of viral or parasitic function within a cell that are only essential when that cell is infected. Since some viral infection result in the induction of survival factors (such as CrrnA, p35) it is likely that at least some cell functions are different and potentially selectively needed during viral, parasite growth.

Another type of screening method means is for small molecules that alter the expression of a specific factor that can be measured and this measurement can be adapted for a screen. This factor can be anything that is accessible to measurement, including but not limited to, secreted molecules, cell surface molecules, soluble and insoluble molecules, binding activities, activities that induce activities on other cells or induce other organic or inorganic chemical reactions. These interactions can be detected by Time Resolved Fluorescence, Surface Plasmon Resonance, Scintillation Proximity Assays, autoradiography, Fluorescence Activated Cell Sorting, or other methods.

Still another screening method is for changes in cell structure that are detected by any means that could be adapted for a selection scheme. This includes, but is not limited to, morphological changes that are measured by physical methods such as differential sedimentation, differential light scattering, differential buoyant density, differential cell volume selected by sieving, atomic force microscopy, electron microscopy.

When screening for bioactivity of test compounds, intracellular second messenger generation can be measured directly. Such embodiments are useful where, for example, the arrayed library is being screened for test compounds which activate or inactivate a particular signaling pathway. A variety of intracellular effectors have been identified as being receptor- or ion channel-regulated, including adenylyl cyclase, cyclic GMP, phosphodiesterases, phosphoinositidases, phosphoinositol kinases, and phospholipases, as well as a variety of ions.

In one embodiment, the GTPase enzymatic activity by G proteins can be measured in plasma membrane preparations by determining the breakdown of y³²P GTP using techniques that are known in the art (For example, see Signal Transduction: A Practical Approach. G. Milligan, Ed. Oxford University Press, Oxford England). When receptors that modulate cAMP are tested, it will be possible to use standard techniques for cAMP detection, such as competitive assays which quantitate [³H]cAMP in the presence of unlabelled cAMP.

Certain receptors and ion channels stimulate the activity of phospholipase C which stimulates the breakdown of phosphatidylinositol 4,5, bisphosphate to 1,4,5-IP3 (which mobilizes intracellular Ca++) and diacylglycerol (DAG) (which activates protein kinase C). Inositol lipids can be extracted and analyzed using standard lipid extraction techniques. DAG can also be measured using thin-layer chromatography. Water soluble derivatives of all three inositol lipids (IP1, IP2, IP3) can also be quantitated using radiolabelling techniques or HPLC.

The other product of PIP2 breakdown, DAG can also be produced from phosphatidyl choline. The breakdown of this phospholipid in response to receptor-mediated signaling can also be measured using a variety of radiolabelling techniques.

The activation of phospholipase A2 can easily be quantitated using known techniques, including, for example, the generation of arachadonate in the cell.

In various cells, e.g., mammalian cells, specific proteases are induced or activated in each of several arms of divergent signaling pathways. These may be independently monitored by following their unique activities with substrates specific for each protease.

In the case of screening for ligands to certain receptors and ion channels, it may be desirable to screen for changes in cellular phosphorylation. Such assay formats may be useful when the host cell expresses a receptor of interest, such as a receptor kinase or phosphatase, and the arrayed library is being screened for peptide sequences which can act in an autocrine fashion. For example, immunoblotting (Lyons and Nelson (1984) Proc. Natl. Acad. Sci. USA 81:7426-7430) using anti-phosphotyrosine, anti-phosphoserine or abti-phosphothreonine antibodies. In addition, tests for phosphorylation could be also useful when the receptor itself may not be a kinase, but activates protein kinases or phosphatase that function downstream in the signal transduction pathway.

In yet another embodiment, the signal transduction pathway of the targeted receptor or ion channel upregulates expression or otherwise activates an enzyme which is capable of modifies a substrate which can be added to the cell. The signal can be detected by using a detectable substrate, in which case lose of the substrate signal is monitored, or altenatively, by using a substrate which produces a detectable product. In preferred embodiments, the conversion of the substrate to product by the activated enzyme produces a detectable change in optical characteristics of the test cell, e.g., the substrate and/or product is chromogenically or fluorogenically active. In an illustrative embodiment the signal transduction pathway causes a change in the activity of a proteolytic enzyme, altering the rate at which it cleaves a substrate peptide (or simply activates the enzyme towards the substrate). The peptide includes a fluorogenic donor radical, e.g., a fluorescence emitting radical, and an acceptor radical, e.g., an aromatic radical which absorbs the fluorescence energy of the fluorogenic donor radical when the acceptor radical and the fluorogenic donor radical are covalently held in close proximity. See, for example, U.S. Pat. Nos. 5,527,681, 5,506,115, 5,429,766, 5,424,186, and 5,316,691; and Capobianco et al. (1992) Anal Biochem 204:96-102. For example, the substrate peptide has a fluorescence donor group such as 1-aminobenzoic acid (anthranilic acid or ABZ) or aminomethylcoumarin (AMC) located at one position on the peptide and a fluorescence quencher group, such as lucifer yellow, methyl red or nitrobenzo-2-oxo-1,3-diazole (NBD), at a different position near the distal end of the peptide. A cleavage site for the activated enzyme will be diposed between each of the sites for the donor and acceptor groups. The intramolecular resonance energy transfer from the fluorescence donor molecule to the quencher will quench the fluorescence of the donor molecule when the two are sufficiently proximate in space, e.g., when the peptide is intact. Upon cleavage of the peptide, however, the quencher is separated from the donor group, leaving behind a fluorescent fragment. Thus, activation of the enzyme results in cleavage of the detection peptide, and dequenching of the fluorescent group.

In a preferred embodiment, the enzyme which cleaves the detection peptide is one which is endogenous to the host cell. For example, the barl gene of yeast encodes a protease, the expression of which is upregulated by stimulation of the yeast pheromone pathway. Thus, host cells which have been generated to exploit the pheromone signal pathway for detection can be contacted with a sutable detection peptide which can be cleaved by barl to release a fluorogenic fragment, and the level of barl activity thus determined.

In still other embodiments, the detectable signal can be produced by use of enzymes or chromogenic/fluorscent probes whose activities are dependent on the concentration of a second messenger, e.g., such as calcium, hydrolysis products of inositol phosphate, cAMP, etc. For example, the mobilization of intracellular calcium or the influx of calcium from outside the cell can be measured using standard techniques. The choice of the appropriate calcium indicator, fluorescent, bioluminescent, metallochromic, or Ca++-sensitive microelectrodes depends on the cell type and the magnitude and time constant of the event under study (Borle (1990) Environ Health Perspect 84:45-56). As an exemplary method of Ca++detection, cells could be loaded with the Ca++ sensitive fluorescent dye fura-2 or indo-1, using standard methods, and any change in Ca++ measured using a fluorometer.

As certain embodiments described above suggest, the signal transduction activity for which an agonist or antagonist is sought in the arrayed library can be measured by detection of a transcription product, e.g., by detecting transcriptional activation (or repression) of an indicator gene(s). Detection of the transcription product includes detecting the gene transcript, detecting the product directly (e.g., by immunoassay) or detecting an activity of the protein (e.g., such as an enzymatic activity or chromogenic/fluorogenic activity); each of which is generally referred to herein as a means for detecting expression of the indicator gene. The indicator gene may be an unmodified endogenous gene of the host cell, a modified endogenous gene, or a part of a completely heterologous construct, e.g., as part of a reporter gene construct.

In one embodiment, the indicator gene is an unmodified endogenous gene. For example, the instant method can rely on detecting the transcriptional level of such endogenous genes as the c-fos gene (e.g., in mammalian cells) or the Bar1 or Fus1 genes (e.g., in yeast cells) in response to such signal transduction pathways as originating from G protein coupled receptors.

In certain instances, it may be desirable to increase the level of transcriptional activation of the endogenous indicator gene by the signal pathway in order to, for example, improve the signal-to-noise of the test system, or to adjust the level of response to a level suitable for a particular detection technique. In one embodiment, the transcriptional activation ability of the signal pathway can be amplified by the overexpression of one or more of the proteins involved in the intracellular signal cascade, particularly enzymes involved in the pathway. For example, increased expression of Jun kinases (JNKs) can potentiate the level of transcriptional activation by a signal in an MEKK/JNKK pathway. Likewise, overexpression of one or more signal transduction proteins in the yeast pheromone pathway can increase the level of Fus1 and/or Bar1 expression. This approach can, of course, also be used to potentiate the level of transcription of a heterologous reporter gene as well.

In other embodiments, the sensitivity of an endogenous indicator gene can be enhanced by manipulating the promoter sequence at the natural locus for the indicator gene. Such manipulation may range from point mutations to the endogenous regulatory elements to gross replacement of all or substantial portions of the regulatory elements. In general, manipulation of the genomic sequence for the indicator gene can be carried out using techniques known in the art, including homologous recombination.

In still another embodiment, a heterologous reporter gene construct can be used to provide the function of an indicator gene. Reporter gene constructs are prepared by operatively linking a reporter gene with at least one transcriptional regulatory element. If only one transcriptional regulatory element is included it must be a regulatable promoter. At least one the selected transcriptional regulatory elements must be indirectly or directly regulated by the activity of the selected cell-surface receptor whereby activity of the receptor can be monitored via transcription of the reporter genes.

Many reporter genes and transcriptional regulatory elements are known to those of skill in the art and others may be identified or synthesized by methods known to those of skill in the art.

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368); β-lactamase or GST.

Transcriptional control elements for use in the reporter gene constructs, or for modifying the genomic locus of an indicator gene include, but are not limited to, promoters, enhancers, and repressor and activator binding sites. Suitable transcriptional regulatory elements may be derived from the transcriptional regulatory regions of genes whose expression is linked to the desired phenotype sought from the arrayed library.

In the case of receptors which modulate cyclic AMP, a transcriptional based readout can be constructed using the cyclic AMP response element binding protein, CREB, which is a transcription factor whose activity is regulated by phosphorylation at a particular serine (S133). When this serine residue is phosphorylated, CREB binds to a recognition sequence known as a CRE (cAMP Responsive Element) found to the 5′ of promotors known to be responsive to elevated cAMP levels. Upon binding of phosphorylated CREB to a CRE, transcription from this promoter is increased.

Phosphorylation of CREB is seen in response to both increased cAMP levels and increased intracellular Ca levels. Increased cAMP levels result in activation of PKA, which in turn phosphorylates CREB and leads to binding to CRE and transcriptional activation. Increased intracellular calcium levels results in activation of calcium/calmodulin responsive kinase II (CaM kinase II). Phosphorylation of CREB by CaM kinase II is effectively the same as phosphorylation of CREB by PKA, and results in transcriptional activation of CRE containing promotors.

Therefore, a transcriptionally-based readout can be constructed in cells containing a reporter gene whose expression is driven by a basal promoter containing one or more CRE. Changes in the intracellular concentration of Ca++ (a result of alterations in the activity of the receptor upon engagement with a ligand) will result in changes in the level of expression of the reporter gene if: a) CREB is also co-expressed in the cell, and b) either an endogenous or heterologous CaM kinase phosphorylates CREB in response to increases in calcium or if an exogenously expressed CaM kinase II is present in the same cell. In other words, stimulation of PLC activity may result in phosphorylation of CREB and increased transcription from the CRE-construct, while inhibition of PLC activity may result in decreased transcription from the CRE-responsive construct.

In preferred embodiments, the reporter gene is a gene whose expression causes a phenotypic change which is screenable or selectable. If the change is selectable, the phenotypic change creates a difference in the growth or survival rate between cells which express the reporter gene and those which do not. If the change is screenable, the phenotype change creates a difference in some detectable characteristic of the cells, by which the cells which express the marker may be distinguished from those which do not. Selection is preferable to screening in that it can provide a means for amplifying from the cell culture those cells which express a test polypeptide which is a receptor effector.

The marker gene is coupled to the receptor signaling pathway so that expression of the marker gene is dependent on activation of the receptor. This coupling may be achieved by operably linking the marker gene to a receptor-responsive promoter. The term “receptor-responsive promoter” indicates a promoter which is regulated by some product of the target receptor's signal transduction pathway.

Alternatively, the promoter may be one which is repressed by the receptor pathway, thereby preventing expression of a product which is deleterious to the cell. With a receptor repressed promoter, one screens for agonists by linking the promoter to a deleterious gene, and for antagonists, by linking it to a beneficial gene. Repression may be achieved by operably linking a receptor-induced promoter to a gene encoding mRNA which is antisense to at least a portion of the mRNA encoded by the marker gene (whether in the coding or flanking regions), so as to inhibit translation of that mRNA. Repression may also be obtained by linking a receptor-induced promoter to a gene encoding a DNA binding repressor protein, and incorporating a suitable operator site into the promoter or other suitable region of the marker gene.

In the case of yeast, suitable positively selectable (beneficial) genes include the following: URA3, LYS2, HIS3, LEU2, TRP1; ADE1,2,3,4,5,7,8; ARG1, 3, 4, 5, 6, 8; HIS1, 4, 5; ILV1, 2, 5; THR1, 4; TRP2, 3, 4, 5; LEU1, 4; MET2,3,4,8,9,14,16,19; URA1, 2,4,5,10; HOM3,6; ASP3; CHO1; ARO 2, 7; CYS3; OLE1; INO1,2,4; PRO1,3 Countless other genes are potential selective markers. The above are involved in well-characterized biosynthetic pathways. The imidazoleglycerol phosphate dehydratase (IGP dehydratase) gene (HIS3) is preferred because it is both quite sensitive and can be selected over a broad range of expression levels. In the simplest case, the cell is auxotrophic for histidine (requires histidine for growth) in the absence of activation. Activation leads to synthesis of the enzyme and the cell becomes prototrophic for histidine (does not require histidine). Thus the selection is for growth in the absence of histidine. Since only a few molecules per cell of IGP dehydratase are required for histidine prototrophy, the assay is very sensitive.

The marker gene may also be a screenable gene. The screened characteristic may be a change in cell morphology, metabolism or other screenable features. Suitable markers include beta-galactosidase (Xgal, C₁₂FDG, Salmon-gal, Magenta-Gal (latter two from Biosynth Ag)), alkaline phosphatase, horseradish peroxidase, exo-glucanase (product of yeast exbl gene; nonessential, secreted); luciferase; bacterial green fluorescent protein; (human placental) secreted alkaline phosphatase (SEAP); and chloramphenicol transferase (CAT). Some of the above can be engineered so that they are secreted (although not β-galactosidase). A preferred screenable marker gene is beta-galactosidase; yeast cells expressing the enzyme convert the colorless substrate Xgal into a blue pigment. Again, the promoter may be receptor-induced or receptor-inhibited.

VII. Exemplary Embodiments

In a particular embodiment, the small molecule array is a small moleculemicroarray which comprises a surface, such as a slide or other flat surface, having affixed thereto a test compound or test compounds in discrete, defined locations at high density (at a large number of locations per unit area, such as at least 1 per cm2 or from about 1 per cj2 to about 1,000,000 per cm2). The small molecule(s) is affixed to the surface (e.g., slide) by means of a hydrogel, combination of hydrogels, biodegradable polymer or combination of biodegradable polymers, from which the small molecule is released under the conditions in which the small molecule microarray is used. In the small molecule array, such as the small molecule microarray, of the present invention, the small molecule-containing locations contain a small molecule or combination of small molecules to be assessed for their effect(s) on at least one (one or more) observable characteristic of a biological sample, such as a phenotypic characteristic of cells and are spaced sufficiently apart that the location are each separate from one another. Each location on a small molecule array can contain the same small molecule or combination of small molecules. Alternatively, different small molecules can be arrayed on a single small molecule array (e.g., each location can contain a different small molecule or small molecule combination or two or more different small molecules or small molecule combinations can be arrayed).

In one embodiment, a small molecule to be assessed for its effect(s) on a phenotypic characteristic of a cell is affixed to a surface, such as a glass or plastic slide, in a hydrogel or combination of two or more hydrogels. Any hydrogel can be used in this embodiment, provided that it can be used to affix (immobilize) small molecules to the surface used; releases small molecules at an appropriate rate; permits cells to attach to it and is not toxic to the cells used. In this embodiment, a small molecule to be assessed for its effect(s) on phenotypic characteristic of a cell is affixed to a surface, such as a glass or plastic slide, in a hydrogel, such as a methacrylate-based polymer, from which the test small molecule is released when in contact with cells placed (plated) onto the small molecule-containing locations. Alternatively, the hydrogel can be, for example, a polycarboxylic acid, cellulosic polymer, polyvinylpyrrolidone, maleic anhydride polymer, polyamide, polyvinyl alcohol or polyethylene oxide.

In a specific embodiment, a small molecule array is produced as follows: A methyacrylate-based mixture, such as one produced as described in the Exemplification, and containing a test small molecule(s) is produced; arrayed on a surface in discrete, defined locations (e.g., as individual spots or drops); and subjected to conditions under which polymerization of the solution occurs, causing the drops to become affixed to the surface (e.g., as test small molecule-containing hydrogel spots). Polymerization can be carried out, for example, by irradiating the drops arrayed on the surface (e.g., with UV light) in an inert nitrogen atmosphere for sufficient time (e.g., 0.5 to 30 minutes) for polymerization to occur. As a result, hydrogel spots form from the drops; the spots remain affixed to the surface in defined, separate locations, thereby producing a small molecule array.

The same procedure is carried out, using smaller quantities of test small molecule-containing hydrogel per location, to produce a small molecule microarray that is a surface that bears a large number of discrete, defined locations or droplets containing test small molecule (a large number of locations per unit area/a density of a least approximately 1 hydrogel spots per cm2). For example, a methacrylate-based mixture containing a test small molecule (or combination of test small molecules) is produced and arrayed on a surface, (e.g., a glass or plastic slide) in individual locations (e.g., as droplets placed in discrete, defined locations), to produce a surface bearing the test small molecule-containing methacrylate-based mixture. Polymerization of the mixture is carried out, such as by subjecting the surface bearing the mixture to irradiation (e.g., by exposure to UV light for an appropriate time, such as from about 0.5 minutes to about 15 minutes), thereby producing a surface bearing test small molecule-containing hydrogel locations, which are gel-like. In this embodiment, the surface bears a large number of hydrogel droplets containing test small molecule (a large number of droplets per unit areas/a density of approximately 1 to 1,000,000 hydrogel spots per cm2). In specific embodiments, the surface bears from about 10 to about 1,000,000 spots per cm2; from about 1,000 to about 1,000,000 spots per cm2; from about 10,000 to about 1,000,000 spots per cm2; from about 100,000 to about 1,000,000 spots per cm2; from about 100 to about 1,000,000 spots per cm2; or any other density of spots that is useful (e.g., from about 50 to about 1,000,000 spots per cm2; from about 500 to about 1,000,000 spots per cm2; from about 500 to about 1,000,000 spots per cm2; from about 5,000 to about 1,000,000 spots per cm2; from about 50, 000 to about 1,000,000 spots per cm2; from about 500,000 to about 1,000,000 spots per cm2. The density of spots on a particular array or microarray will be determined by such factors as the test small molecule(s) to be assessed and the cell type used and will be determined empirically, using known methods.

The surface used to produce such small molecule arrays (e.g., small molecule arrays, small molecule microarrays) can be any surface to which hydrogels can be affixed and remain under the conditions under which an assay is carried out, such as conditions under which a cell-based assay is carried out. The surface can be a non-porous solid support of a porous solid support. It can be, for example, glass, plastic (e.g., polytetrafluoroethylene, polyvinylidencflouride, polystyrene, polycarbonate, polypropylene), minerals (e.g., hydrozylapatite or graphite), metal (e.g., gold) or membranes, such as nitrocellulose, methylcellulose, PTFE or cellulose. The surface can comprise low volume nanowells (wells of less than or equal to 1 microliter). The test small molecule-hydrogel mixture can be deposited in the bottom of a nanowell using, for example, a pin device, such as a microarray spotter. The surface can also be a patterned surface that comprises alternating regions of hydrophobic and hydrophilic surfaces that permit cell growth only on the hydrophilic areas.

In a second embodiment of the small molecule array of the present invention, a test small molecule or test small molecules is affixed to the surface by means of a biodegradable polymer or combination of two or more biodegradable polymers. Any biodegradable polymer can be used provided that it can be used to affix (immobilize) small molecules to the surface used; releases small molecules at an appropriate rate; permits cells to attach to it and is not toxic to cells.

In this embodiment, a small molecule to be assessed for its effect(s) on a phenotypic characteristic of a cell is affixed to a surface, such as a glass or plastic slide, in discrete, defined locations in a biodegradable polymer, such as, but not limited to, poly(lactic acid), poly (glycolic acid), poly lactide coglycolide and gelatin. Cells placed onto the small molecule-containing locations biodegrade the polymer, resulting in release of the test small molecule, which comes in contact with and/or enters into the cells, making it possible to assess the effect(s) of the small molecule(s) on an observable phenotypic characteristic(s) of the cells.

In a specific embodiment, a small molecule array is produced as follows: A biodegradable polymer containing a test small molecule or small molecules is produced; arrayed on a surface in discrete, defined locations (e.g., as individual spots or drops); and subjected to conditions under which polymerization occurs, causing the drops to become affixed to the surface (e.g., as test small molecule-containing biodegradable polymer spots). As a result, test small molecule-containing biodegradable polymer spots form from the drops; the spots remain affixed to the surface in defined, separate locations, thereby producing a small molecule array.

The same procedure is carried out, in one embodiment, to produce a small molecule microarray on which test small molecule-containing biodegradable polymer spots are arrayed. For example, a mixture comprising at least one biodegradable polymer and a test small molecule (or slide) in individual locations (e.g., as droplets placed in discrete, defined locations), to produce a surface bearing the mixture in discrete, define (separate) locations. Polymerization of the mixture is carried out, such as by heating or drying thereby producing a surface bearing test small molecule-biodegradable polymer locations, which are gel-like. In this embodiment, the surface bears a large number of discrete, defined locations or droplets containing test small molecule (a large number of locations per unit area/a density of approximately 1 to 1,000,000 gel-like spots per cm2).

The surface used to produce small molecule arrays (e.g., small molecule arrays, small molecule microarrays) can be any surface to which a biodegradable polymer can be affixed and remain under the conditions under which an assay is carried out, such as conditions under which a cell-based assay is carried out. The surface can be a non-porous solid support of a porous solid support. It can be, for example, glass, plastic (e.g., polytetrafluoroethylene, polyvinylideneflouride, polystyrene, polycarbonate, polypropylene), silicon, minerals (e.g., hydroxylapatite or graphite), metal (e.g., gold) or membranes, such as nitrocellulose, methylcellulose, PTFE or cellulose or any of these surfaces coated with a compound (e.g., polymer, small molecule, protein, metal ion, oligonucleotide, peptide) that promotes adhesion of the spots to the surface (e.g., polylysine). The surface can comprise low volume nanowells (wells of less than or equal to 1 microliter). The test small molecule-biodegradable or hydrogel or other polymer mixture can be deposited in the bottom of a nanowell using, for example, a pin device, such as a microarray spotter. The surface can also be a patterned surface that comprises alternating regions of hydrophobic and hydrophilic surfaces that permit cell growth only on the hydrophilic areas.

A further embodiment of the present invention is arrays, such as microarrays, that can be used to identify small molecules that modulate (inhibit or enhance, including activativation of or increase in) enzyme activity. In this embodiment, arrays are made as described above, thereby producing hydrogel-based arrays or biodegradable polymer-based arrays (microarrays) which comprise test small molecules to be assessed for their effect(s) on enzyme activity. An enzyme of interest (an enzyme for which an inhibitor or enhancer is sought) and a substrate of the enzyme are added to the test small molecule-containing microarray surface, sequentially or simultaneously, thereby producing a microarray surface bearing test small molecules, enzyme of interest and enzyme substrate. The enzyme and enzyme substrate are in solution or other appropriate carrier; they can be present in the same solution or carrier or in separate solutions or carriers. In one embodiment, the enzyme substrate of the enzyme of interest becomes insoluble and precipitates from solution when it is acted upon by the enzyme. As a result, locations that contain test small molecules that inhibit or enhance enzyme activity can be identified by a decrease in precipitate or an increase in precipitate at those locations, respectively, relative to an appropriate control. For example, if a test small molecule at a location inhibits enzyme activity, less precipitate will be produced at that location than at a control location (e.g., a location that lacks the test small molecule and is maintained under the same conditions as the test small molecule-containing location). Alternatively, if a test small molecule at a location enhances enzyme activity (activates the enzyme or increases its activity), more precipitate is formed at that location than at a control location (e.g., a location that lacks the test small molecule is maintained under the same conditions as the test small molecule-containing location). In one embodiment, the peroxidase reaction or the ELF fluorescent substrate (Molecular Probes) is used. See <http://www.probes.coni/handbook/sections.0602.html>. Also see manufacturer's description: “Our patented ELF 97 phosphate is an alkaline phosphatase substrate with several unique properties that make it superior to many of the existing reagents for these applications. Upon enzymatic cleavage, this weekly blue-fluorescent substrate yields a bright yellow-green-fluorescent precipitate that exhibits an unusually large Stokes shift and excellent photostability. The ELF 97 phosphatase substrate is a particularly powerful tool for immunohistochemistry, MRNA in situ hybridization methods, and detection of DNA on DNA “chips”. Unlike the radioactive signal produced by conventional methods, ELF 97 mRNA detection signals can be developed in minutes or even seconds and can be clearly distinguished from sample pigmentation, which often obscures both radioactive and calorimetric signals. Moreover, in this application, the yellow-green-fluorescent precipitate of the ELF 97 alcohol produces a signal that is many-fold brighter than that achieved when using either directly labeled fluorescent hybridization probes or fluorescent secondary detection methods.”

A further embodiment is arrays, such as microarrays, that can be used to identify small molecules that modulate (decrease or enhance) protein binding activity. In this embodiment, arrays are made as described herein, thereby producing hydrogel-based arrays (microarrays_ or biodegradable polymer-based arrays (microarrays) which comprise test small molecules to be assessed for their effect(s) on protein binding activity. In this embodiment, the hydrogel or biodegradable polymer mixture comprises a reagent, such as an antibody, streptavidin, collagen, nickel chelate, that allows one or more proteins to adhere to the test small molecule-containing hydrogel spots or test small molecule-containing biodegradable polymer spots formed by polymerization on the microarray surface. The resulting microarray, referred to herein as a protein binding activity assessment microarray, comprises, in discrete, defined locations, test small molecule-containing hydrogel spots or test small molecule-containing biodegradable polymer spots that additionally comprise a reagent that allows one or more proteins to adhere to the spots on the microarray surface. A protein for which a small molecule that modulates binding of the protein to a binding partner is sought (a test protein) and a binding partner can be added sequentially or simultaneously, thereby producing a microarray surface bearing test small molecule(s), protein of interest, and a binding partner of the protein of interest. The protein of interest and its binding partner are in solution or other appropriate carrier; they can be present in the same solution or carrier or in separate solutions or carriers. The binding partner or cause displacement of a binding partner from the location, can be identified by decreased (partially or totally) intensity of signal from the labeled binding partner, relative to an appropriate control. For example, if a test small molecule at a location inhibits binding activity of a protein of interest, signal from the labeled partner (e.g., fluorescently labeled binding partner) will be less at that location than at a control location (e.g., location that lacks the test small molecule and is maintained under the same conditions as the test small molecule-containing location). Alternatively, if a test small molecule at a location enhances protein binding activity (e.g., increases the avidity or specificity of binding), signal is greater at that location than at a control location (e.g., a location that lacks the test small molecule and is maintained under the same conditions as the test small molecule-containing location).

An additional embodiment is a microarray useful to identify compounds or molecules (small molecules) that modulate (decrease or enhance) an activity of interest in a cell lysate. In this embodiment, microarrays are produced as described herein, thereby producing hydrogel-based arrays (microarrays) or biodegradable polymer-based arrays (microarrays) which comprise test small molecules to be assessed for their effect(s) on activity of cell lysates. A cell lysate that exhibits an activity of interest (e.g., RNA of protein

synthesis, protein phosphorylation, protein degradation, protein phosphorylation or protein glycosylation) and a substance for the activity of interest are added to the test small molecule-containing microarray surface, sequentially or simultaneously, thereby producing a microarray surface bearing test small molecules, cell lysate and a substrate for the activity of interest. The cell lysate and substrate for the activity of interest are in solution or other appropriate carrier; they can be present in the same solution or carrier or in separate solutions or carriers. In one embodiment, the substrate becomes insoluble and precipitates from solution when it is acted upon by the activity of interest of the cell lysate. As a result, locations that contain test small molecules that modulate (inhibit or enhance) the activity of interest of the cell lysate can be identified by a decrease in precipitate or an increase in precipitate at those locations, respectively, relative to an appropriate control. For example, if a test small molecule at a location inhibits the activity of interest, less precipitate will be produced at that location than at a control location (e.g., a location that lacks the test small molecule and is maintained under the same conditions as the test small molecule-containing location). Alternatively, if a test small molecule at a location enhances the activity of interest, more precipitate is formed at that location than at a control location (e.g., a location that lacks the test small molecule and is maintained under the same conditions as the test small molecule-containing location). In one embodiment, the peroxidase reaction or the ELF fluorescent substrate (Molecular Probe) is used.

A further embodiment of the present invention is microarrays useful to identify small molecules that that modulate (decrease or enhance) an activity of interest in a cell lysate. In this embodiment, microarrays are produced as described herein, thereby producing hydrogel-based arrays (microarrays) or biodegradable polymer-based arrays (microarrays) which comprise test small molecules to be assessed for their effect(s) on activity of tissue slices. A tissue slice, such as a tissue slice of from about 1 μm to about 10,000 μm in thickness is placed on the small molecule array and the resulting array bearing the tissue slice is incubated for sufficient time (e.g., 0.1 to 1000 hours) for the small molecules to exert effects on the tissue slice. The effect, if any, of a small molecule is determined by detecting changes that occur in an observable property or properties of the tissue slice. Locations which contain test small molecules that modulate (decrease or enhance) an observable property of the tissue slice (e.g., subcellular migration of a protein, cell death or cell morphology, increase or decrease in protein or RNA expression) can be identified by the decrease in or enhancement of that property, relative to an appropriate control.

An alternative embodiment of the present invention is microarrays useful to identify compounds or molecules (small molecules) that modulate (decrease or enhance) an activity of interest in a small organism, such as yeast cells. In this embodiment, microarrays are produced as described herein, thereby producing hydrogel-based arrays (microarrays) or biodegradable polymer-based arrays (microarrays) which comprise small molecules to be assessed for their effect(s) on activity of yeast cells. An agar layer containing nutrients sufficient for (that support) yeast cell growth is poured on top of the microarray and allowed to solidify. The layer is typically from about 10 μm to about 10,000 μm thick. Yeast cells are placed on top of the agar layer in medium that contains nutrients sufficient for growth. The resulting array, which comprises the test small molecule-containing locations, agar and yeast in medium, is maintained under conditions that result in evaporation of the medium and permit sufficient time for changes in an observable property (e.g., growth, differentiation, or expression of a protein of interest) to occur. Test small molecules that modulate (enhance or inhibit) the observable property are detected by noting a difference between that characteristic in yeast cells at locations that contain the test small molecule (test yeast cells) and an appropriate control (e.g., the same type of yeast cells maintained under the same conditions but in the absence of test small molecule). Alternatively, microarrays are made as described herein except that the discrete, defined locations comprise agar and nutrients required for yeast cell growth. Yeast cells in medium that contains nutrients sufficient for their growth are placed on top of the array and the resulting microarray, which comprises test small molecule-containing locations, agar and yeast in medium, is maintained under conditions that result in evaporation of the medium and permit sufficient time for changes in an observable property (e.g., growth or change in protein or rnPNA expression) to occur. Test small molecules that modulate the observable property are detected as described immediately above.

Alternatively, a microarray of the present invention is useful to identify small molecules that alter an observable property of a small organism, such as C.elegans. In this embodiment, microarrays are produced as described herein, thereby producing hydrogel-based arrays (microarrays) or biodegradable polymer-based arrays (microarrays) which comprise test small molecules to be assessed for their effect(s) on C. clegans. An agar layer containing nutrients sufficient for (that support) growth of C. elegans is placed on top of the microarray and allowed to solidify. The layer is typically from about 10 μm to about 10,000 μm thick. Worms are placed on top of the agar layer in medium containing nutrients sufficient for their growth (e.g., E. coli) and the resulting microarray, which comprises test small molecule-containing locations, agar and worms in medium, is maintained under conditions that result in evaporation of the medium and permit sufficient time for changes in an observable property (e.g., growth or change in protein or MRNA expression) to occur. Test small molecules that modulate (enhance or inhibit) the observable property are detected by noting a difference between that characteristic in C. elegans at locations that contain the test small molecule (test C. elegans) and an appropriate control (e.g., C. elegans maintained under the same conditions but in the absence of test small molecule).

Small molecules can be arrayed on a surface to produce an array of the present invention, such as on the surface of a microarray, using known methods. For example, they can be arrayed by means of an arraying device, such as a pin transfer device, such as the polypropylene pin transfer device described in the Exemplification, or an inkjet printer (piezoelectric pipetting system). It is also possible to array the small molecules manually, using a pipetman, a small pin device, a stamping device or a quill pen.

Small molecule arrays of the present invention can comprise any test small molecule of interest (any small molecule to be assessed for its effects on a biological sample), provided that it can be affixed to a surface in combination with a hydrogel or biodegradable polymer. As used herein, the term small molecule includes any agent (compound or molecule) that affects any process in an organism. Test small molecules include agents (molecules or compounds) that are known small molecules and agents that are not known to have an effect on a process. Test small molecules that are known small molecules (e.g., a small molecule known to be useful in treating hypertension) can be assessed, using arrays of the present invention, for example, to identify additional uses, to determine optimal effective concentrations and to assess interactions (e.g., synergistic effects or adverse effects) with other test small molecules or small molecules. For example, small molecules available from existing libraries, such as commercially-available small molecule libraries or libraries available from a wide variety of government agencies and academic sources, can be used to produce small molecule arrays (e.g., www.chembridge.com; www.chemdiv.com; www.comgencx.com). Alternatively, cell products, obtained from lysed cells, from media or broth in which cells have been cultured, or organic extracts of plants or other organisms can be arrayed on an array, particularly a microarray., of the present invention. Such cells products can be obtained or isolated from cells, broth or media by known methods, such as by organic solvent extraction, crystallization, or chromatography. See, for example, Sambrook, J. et al, Molecular Cloning (2^{nd} edition), Cold Spring Harbor Press (1989); Ausubel, F. M. et al, Current Protocols in Molecular Biology, Green Publishing Association and Wiley-Interscience (1988). Examples of test small molecules that can be assessed include, but are not limited to, agents to be assessed for their effects on cell size, proliferation (e.g., rate, extent), viability, DNA composition, expression of mRNAs and proteins (specifically or nonspecifically), metastatic capability, and post-translational modification of cellular components, such as specific macromolecules (e.g., acetylated histones, phosphorylated proteins, glycosylated proteins, methylated DNA).

The effects of test small molecules can be assessed on any type of cell of interest. For example, test small molecules can be assessed on any type of cell of interest. For example, test small molecules can be assessed for their effects on human cells (e.g., hair follicles; cancer cells from solid tumors or soft tissues (leukemias, lymphomas); bone marrow cells; bone cells; stem cells; cells from liver, kidney, heart, brain, muscle, skin, spleen, gastrointestinal tract and other organs and tissues) and those shown to have a desired effect can be administered (as identified or after appropriate modification, if needed) to an individual, such as a human, in need of the effect shown. Alternatively, test small molecules can be assessed for their effects on other cell types, such as vertebrate cells (e.g., primate and nonprimate, including monkeys, gorillas, domestic animals, farm animals and HeLaS3 cells, A549 lung carcinoma cells, 293 human embryonic kidney cells, PC12 cells) and nonvertabrate cells (e.g., Schneider cells, Sf9 insect cells). Similarly, they can be assessed for their effects on bacteria, viruses, parasites, prions, and fungi. Cells can be adherent cells or nonadherent cells, provided that in the latter case, the cells, hydrogel or biopolymer and/or array surface have been modified or engineered in such a manner that the cells adhere to the small molecule-containing locations under the conditions used. Non-adherent cells can be altered or modified (e.g., by expressing macrophage scavenger receptor or an antibody against a specific protein present on the microarray spots) in order to render them adherent (to engineer adhesiveness into them). Alternatively, a surface to which non-adherent cells are to be affixed can bear (have attached thereto) a moiety, such as an antibody that recognizes (binds) a surface a membrane protein of the cell type to be affixed to the surface. Cells can be unmodified (used as they are obtained) or modified, such as by genetic engineering or being subjected to a mutagenizing agent (e.g., chemical, radiation).

In those instances in which a control is used, the control can be, for example, the same type of biological sample (e.g., the same type of cell, tissue or organism) as the test sample (the sample contacted with the test small molecule-containing locations); the corresponding normal or wild type cell type (e.g., normal cells corresponding to or of the same origin as cancerous cells) or any other appropriate control. Controls are treated in the same manner as test samples (samples contacted with test small molecule-containing locations) except that they are not contacted with a test small molecule or test small molecules. Controls can be carried out before, at the same time as or after the test samples are assessed, provided that they are treated in the same manner as the test samples except for the presence of a test small molecule or test small molecules. For example, the control can be a predetermined control.

Small molecules identified by use of the arrays of the present invention can be used to treat a wide variety of conditions, such as cancer, hypertension, heart disease, metabolic conditions (e.g., diabetes, weight gain, impotence, psychiatric disorders, spinal cord injuries, infectious diseases, parasitic conditions and hair loss.

Another embodiment of the present invention is a small molecule-cell array, particularly a small molecule-cell microarray, which comprises: (a) at least one small molecule arrayed in defined, discrete (separate) locations on a surface and affixed to the surface by a hydrogel, combination of hydrogels, biodegradable polymer or combination of biodegradable polymers and (b) cells plated thereon. The small molecule(s) can be affixed to the surface, such as a slide or other flat surface, by means of a hydro9gel or biodegradable polymer, as described herein. The cells are plated onto the surface using known methods, such as by covering the entire surface (areas covered by test-small molecule containing hydrogel spots or test small molecule-containing polymer spots, as well as the intervening spaces) and then, optionally, removing cells that do not attach to the geldisks or polymer spots. After the cells are plated onto the surface, thereby producing a small molecule-cell array that comprises a test small molecule arrayed in defined, discrete locations (in test small molecule-containing spots) and cells plated thereon, the small molecule-cell array is maintained under conditions (e.g., temperature, time, humidity and CO₂ environment) appropriate for cells attached to the test small molecule-containing locations and the test small molecule(s) to be released and make contact with cell membranes and/or enter into cells and to produce effect(s) on the cells. Whether the small molecule has an effect on cells is determined by observing a change in at least one observable characteristic of the cells, using any method which makes it possible to detect such changes (e.g., fluorescence (See Current Protocols in Molecular Biology), autoradiography (Ziauddin, J., Sabatini, D. M. (2001) Microarrays of cells expressing defined cDNAs. Nature (London), 411:107-110), chemiluminescence (Stockwell, B. R., Hagarty, S. J., and Schreiber, S. L. Chemistry and Biology (1999) 6:71-83), phase contrast (Basic Cell culture Protocols, 2^{nd} Edition Edited by Jeffrey W. Pollard and John M. Waler. Humana Press, (Totowa, New Jersey, 1997), differential interference contrast (See Current Protocols in Molecular Biology), electron microscopy (Basic Cell culture Protocols, 2^{nd} Edition Edited by Jeffrey W. Pollard and John M. Walker. Humana Press, Totowa, New Jersey, 1997, atomic force microscopy (168.A. T. Woolley, C. L. Cheung, J. H. Hafner and C. M. Lieber, “Structural Biology with Carbon Nanotube AFM Probes” Chem. Biol. 7,R193-R204 (2000), or immunohistochemistry Sambrook, J. et al, Molecular Cloning (2^{nd} edition), Cold Spring Harbor Press (1989); Ausubel, F. M. et al., Current Protocols in Molecular Biology, Green Publishing Association and Wiley-Interscience (1988).

Another embodiment of this invention is a method of identifying a small molecule that has an effect on a phenotypic characteristic of cells. The method comprises (a) observing at least one phenotypic characteristic (observable property) of test cells, wherein the test cells are plated on a small molecule array and wherein the small molecule array comprises the test small molecule arrayed at discrete, defined locations on a surface; (b) comparing the phenotypic characteristic of the test cell with the corresponding phenotypic characteristic of control cells; (c) determining if the phenotypic characteristic of the test cell is different from the corresponding phenotypic characteristic of control cells, wherein if the phenotypic characteristic of the test cell is different from the corresponding phenotypic characteristic of control cells, the test small molecule is a small molecule that affects at least one phentotypic characteristic of the test cells. Control cells are the same type of cells as the test cells are treated in the same way (subjected to the same conditions) as test cells, except that no small molecule is present on the array (control cells are not exposed to or maintained in the presence of small molecule). In one embodiment, the method comprises assessing the effect of a test small molecule on at least one phenotypic characteristic (observable property) of cells, wherein the cells are plated on a small molecule array and wherein the small molecule array comprises the test small molecule arrayed in test small molecule-containing hydrogel spots at discrete, defined locations on a surface. A specific example of this embodiment is a method of identifying a small molecule that has an effect on a phenotypic characteristic of cells that comprises assessing the effect of a test small molecule on at least one phenotypic characteristic (observable property) of cells, wherein the cells are plated on a small molecule array and wherein the small molecule array comprises the test small molecule arrayed in test small molecule-containing hydrogel disks at discrete, defined locations on a surface. A specific example of this embodiment is a method of identifying a small molecule that has an effect on a phenotypic characteristic of cells that comprises assessing the effect of a test small molecule on at least one phenotypic characteristic of cells, wherein the cells are plated on a small molecule microarray and the small molecule microarray comprises the test small molecule arrayed in test small molecule-containing hydrogel disks at discrete, defined locations in large numbers on a surface (e.g., from about 1 to about 1,000,000 locations per cm²). The effect of a test small molecule on a phenotypic characteristic of cells is determined by observing the phenotype of the cells in the presence and in the absence of the test small molecule. For example, cells, referred to as test cells, are cultured on an array, such as a microarray, that comprises small molecule-containing locations; effect(s), if any, on phenotypic characterist9c(s) are observed; and the results are compared with the effect(s), if any, on the corresponding phenotypic characteristic(s) of the same type of cells grown under the same conditions as the test cells but in the absence of the test small molecule (control cells). If there is a difference in the phenotypic characteristic of the test cells and the control cells (e.g., test cells do not proliferate and control cells proliferate; test cells fail to produce a specific protein normally produced by such cells and control cells produce the protein), the test small molecule is a small molecule that affects the phenotypic characteristic.

The method of identifying a small molecule that affects a phenotypic characteristic(s) of a cell can be carried out in order to identify a small molecule that has an effect on one or more preselected (specific phenotypic characteristics of interest of the cell, such as growth, cell size, viability. That is, the small molecule array, such as the small molecule microarray, of the present invention is useful to identify small molecules that affect a pre-selected characteristic(s) of cells. For example, the small molecule arrays and method of the present invention can be used to identify small molecules that inhibit cell growth, size and/or viability. Alternatively, the method of the present invention can be carried out to determine what effect(s), if any, a small molecule has on phenotypic characteristic(s) of cells, without having pre-selected phenotypic characteristic(s) of interest. For example, the method and small molecule assays can be used to determine what effect, if any, a test small molecule, such as a putative toxic compound or molecule (e.g., paclitaxel) has on cells. The method of the present invention can be carried out using ay type of cell and any test small molecule whose effects on cells are of interest, as described herein with reference to the small molecule arrays of the present invention.

Similarly, the method can be carried out to assess the effect(s) of a combination of small molecules (present in the same defined, discrete location or in two or more such locations provided that the locations are sufficiently close that the test small molecules in the multiple locations contact and/or enter cells on which the effect(s) of the combination of small molecules is t o be assessed) on the array of the present invention). The method and small molecule array of the present invention can also be used to determine the effect on a test small molecule on cells that are subjected to an additional condition (e.g., treatment with another small molecule, stimulation with a growth factor, introduction of an environmental stress such as heat or introduction of peptides or proteins, DNA or RNA). For example, cells, such as lung cells (test cells), can be plated on a small molecule array, such as a small molecule microarray, on which small molecules to be assessed for their protective effect against radiation, toxic fumes or cigarette smoke are arrayed and the resulting small molecule-cell array can be maintained under conditions appropriate for growth and proliferation of the cells and subjected to the additional condition (e.g., subjected to radiation, grown in the presence of toxic fumes or cigarette smoke). The effects of a small molecule on a phenotypic characteristic(s) of the test cells on the small molecule-cell array (e.g., the effects on lung cells) are determined and compared with the corresponding phenotypic characteristic(s) of an appropriate control. An appropriate control is the same cell type maintained under the same conditions as those under which the test cells are maintained and on an array that is the same as the array on which the test cells are maintained and on an array that is the same as the array on which the test cells are maintained, except that it does not contain test small molecules. Differences in phenotypic characteristics of the test cells and the control cells are noted. Such differences are the result of the effects of a test small molecule in the presence of the additional condition and indicate that the test small molecule is a small molecule that has an effect on or interacts with the additional condition or factor and alters the effect it would otherwise have on the phenotypic characteristic. For example, if test cells, such as cancerous lung cells, are maintained on a small molecule-cell array and subjected to radiation and cells grown on particular small molecule-containing locations are killed at lower doses of radiation than control cells, small molecule(s) at the locations identified are small molecules that facilitate/enhance killing of cancerous lung cells by radiation.

The present invention further encompasses a method of assessing the ability of a small molecule shown by the method of the present invention to affect at least one phenotypic characteristic of cells to affect the same phenotypic characteristic of the same cell type in an animal, such as a human or a non-human animal, such as an animal model. The method comprises (a) observing at least one phenotypic characteristic (observable property) of test cells, wherein the test cells are plated on a small molecule array and wherein the small molecule array comprises the test small molecule arrayed at discrete, defined locations on a surface by means of a polymer that immobilizes the small molecule to the surface, permits release of the small molecule and attachment of cells plated thereon and is not toxic to cells plated thereon; (b) comparing the phenotypic characteristic of the test cells with the corresponding phenotypic characteristic of control cells; (c) determining if the phenotypic characteristic of the test cells is different from the corresponding phenotypic characteristic of control cells, wherein if the phenotypic characteristic of the test cells is different from the corresponding phenotypic characteristic of control cells, the test small molecule is a small molecule that affects at least one phenotypic characteristic of the test cells; (d) administering the small molecule of (c) to an appropriate animal model, referred to as a test animal; (e) maintaining the test animal under conditions appropriate for the small molecule to exert its effect and (f) assessing the effect of the small molecule on the corresponding phenotypic characteristic in the test animal, wherein if the small molecule has substantially similar effect in the test animal as the effect observed in (a), then the small molecule is small molecule that affects the same phenotypic characteristic of the same cell type in an animal. Alternatively, a small molecule identified as having an effect on at least one phenotypic characteristic of test cells can be administered to a test animal and phenotypic characteristics in addition to or other than that observed in test cells can be assessed. The present invention is illustrated by the following exemplification, which is not intended to be limiting in ay way.

### EXEMPLIFICATION

### Example 1

This exemplification describes the combination of a test small molecule with semi-porous polymer, and release of the compound from the polymer on a time-scale compatible with cell-based phenotypic assays, such as the cell death assay used here.

7.2 mg of phenylarsine oxide (Sigma) was dissolved in 6000 microliters of a pre-gel solution (1% Iragucre 651 (Cibe Speciality Chemicals), 8% diethyleneglycol dimethacrylate (Aldrich), 8% methacrylic acid (Aldrich), 15% t-butylaminoethyl methacrylate (Aldrich), 68% 2-hydroxyethyl methacrylate (Aldrich)) to create a 71.4 mM solution of phenylarsine oxide.

The 71.4 mM solution of phenylarsine oxide was serially diluted two-fold into the pre-gel solution to create phenylarsine oxide solution at concentrations 35.7 mM, 17.9 mM, 8.9 mM, 2.2mM, 1.1 mM, 0.55 mM, 0.28 mM.

50 μL of each of these solutions was placed in a single row of a 384-well polypropylene plate (Matrix Technologies, catalog # 4313) as well as a 50 μL of the pre-gel solution lacking phenylarsine oxide as a control.

A polypropylene pin transfer device (Matrix Technologies, catalog # 350500130) was dipped into the solutions in the 384-well plate and then touched against a glass microscope slide (VWR Scientific Products, catalog # 48311-702) to transfer small droplets (i.e., 20-100 nL) of each solution in a regular array onto the microscope slide.

The slide with the arrayed droplets was placed into a T175 flask through a small aperture opposite the neck. This aperture was then sealed with TimeTape (VWR). The flask was flooded for 30 seconds with nitrogen gas (BOC) at 2 psi by piercing a disposable needle through the vented cap to the flask and attaching the needle to a compressed gas cylinder.

With the gas still flowing, a long wave UV lamp (365 nm) was placed on top of the flask and used to irradiate the slide with UV light for 2 minutes. This process caused polymerization of the solution, causing hard gel-like disks to form on the slide.

The slide was removed from the T175 flask and placed in a 10 cm plastic Petri dish.

17 mL of a solution of medium (10% fetal bovine serum (Life Technologies), Dulbecco's Modified Eagle Medium (Life Technologies), 100 units/mL penicillin G sodium (Life Technologies), 1000 μg/mL, streptomycin sulfate (Life Technologies)) containing 10 million A549 human lung carcinoma cells was placed over the slide in the Petri dish.

The dish was incubated in a humidified atmosphere containing 5% CO₂ at 37° C. for 18 hours. The slide was removed from the incubator at this point and observed using a phase-contrast microscope.

It was found that the gel-like disks containing greater than 0.55 mM phenylarsine oxide harbored rounded cells, which is a characteristic of dying cells. The control spot lacking phenylarsine oxide did not affect cell viability, cell number or cell morphology. By counting the number and extent of rounded cells on each spot, the concentration of phenylarsine oxide casing a 50% loss of cell viability was estimated by be 1 mM. Cells not vicinal to the phenylarsine oxide-containing spots were not affected.

### Example 2

This exemplification describes a protocol for preparing a test compound micro array.

All pins are from Cartesian Technologies.

1. Wash protocol

a. Home the axis

b. Move to water bath and submerge the pin bevel well below the surface of the water

c. Shake the pin up and down by 0.01 mm 15 times

d. move to vacuum station and lower pin into hole for 1000 ms

e. repeat steps c-d two more times

f. vacuum pin for an additional 2000 ms

2. Transfer sample from 384well round bottom polypropylene plate

a. Move to position

b. Lower pin into well for 3000 ms

3. Preprint

a. print on a glass slide (VWR plain with no frost 1 mm thick)

b. print for various times and numbers of repetitions

c. when printing with methyl salicylate use Imm spacing for preprints, and 2mm spacing for DMSO solutions.

4. Making array

a. move to position on array print and spot(s) on 1 st slide, then 2nd . . . etc

b. when complete, redo wash without homing of axis

Repeat steps 2-4 for each sample to be spotted

5. “end method” wash is the same as the initial wash except that the robot does not home and the print head moves to the back of the chamber to get it out of the way.

This exemplary protocol may be readily adjusted in a variety of ways, including variations in the following: pin type/size, print time (the time the pin is down on the array slide), preprint number, pre print time, the number of successive prints of the same solution after drying in same place (termed here “X”), drying time (the time spots are left to dry between successive X), printing temperature, humidity and slide type.

### Example 3

This exemplification describes a protocol for coating a test compound microarray slide with fibronectin.

A 1 mg/ml stock is prepared as follows: 1 mg fibronectin (BD Biosciences, cat# 354008, from human plasma, lyophilized) is suspended in 1 ml sterile H20 making sure that the vial has come to RT before adding water (about 45 min). The vial is allowed to sit at room temperature for 45 min and then the mixture is aliquotted into eppendorf tubes for storage at −20C. For use, the stock is thawed on ice with little agitation and then 500 μl of 60 μg/ml solution in serum-free medium is made for each slide (30 μl of stock added to 470 μl of serum-free medium). The mixture is kept on ice until used. The slide is placed in a humidity chamber 500 μl of solution was pipetted onto the slide. A piece of parafilm was placed on top of the slide to evenly spread the coating. Slides were incubated at room temperature for lhr, the parafilm was removed and slides were dipped once in dH2O and let dry at room temperature for 20 min.

### Example 4

This exemplification provides a method for generating a test compound microarray with a PDLA matrix.

1. Making solutions

a. PDLA (25,000 MW) is dissolved in methyl Salicylate (MS) at 20 mg/ml

b. Drugs are dissolved at 2X final concentration in MS

c. Mix the two solutions creating a IX drug solution at 10 mg/ml at RT and pipette 15 ul of each into 384well plate (round bottom polypropylene)

2. Printing:

a. Print solutions with the following parameters:

| | |
|---|---|
| pin type/size | SMP4 or 10 |
| print time | 50 ms |
| preprint number | 20-40 |
| pre print time | 200 ms |
| “X” | 1-5 X |
| drying time | 10 min |
| printing temp: | 27 C. |
| humidity: | 55% |
| slide type: | Nickel chelated from Xenopore |

b. Let slides dry at RT for at least 20 min

3. Coat slides according to the fibronectin coating protocol (See Example 3)

4. Place slides in either a 10 cm round or square petri dish adding 8e6 cells in 25 mls or

6.5e6 cells in 15 mls for each respectively

5. Incubate at 37C in CO2 for 14 hours

### Example 5

This exemplification provides a method for generating a test compound microarray with a PDLAG matrix.

1. Making solutions

a. PDLAG is dissolved in methyl Salicylate (MS) at 200 mg/ml

b. Drugs are dissolved at 2X final concentration in DMSO

c. Mix the two solutions creating a 1X drug solution at 100 mg/ml polymer at RT and pipette 15 μl of each into 384well plate (round bottom polypropylene). Solutions are only good to print with for around 1 hour before phase separation occurs.

2. Printing:

| | |
|---|---|
| pin type/size | SMP4 or 10 |
| print time | 50 ms |
| preprint number | 20-40 |
| pre print time | 200 ms |
| “X” | 1-5 X |
| drying time | 10 min |
| printing temp: | 27 C. |
| humidity: | 55% |
| slide type: | Nickel chelated from Xenopore |

a. Print solutions with the following perameters:

b. Let slides dry at RT for at least 20 min

3. Coat slides according to the fibronectin coating protocol (see Example 3)

4. Place slides in either a 10 cm round or square petri dish adding 8e6 cells in 25 mls or

6.5e6 cells in 15 mls for each respectively

5. Incubate at 37C in C02 for 14 hours

### Example 6

This exemplification provides an alternate “sandwich” method for generating a test compound microarray with a PDLAG matrix.

1. Making solutions

a. PDLAG is dissolved in methyl Salicylate (MS) at 100 mg/ml

b. Drugs are dissolved at final concentration in DMSO

c. Do NOT mix solutions prior to printing

2. Printing:

constant factors throughout:

| | |
|---|---|
| preprint number | 20-40 |
| pre print time | 200 ms |
| drying time | 10 min |
| printing temp: | 27 C. |
| humidity: | 55% |
| slide type: | Nickel chelated from Xenopore |

a. Print foundation layer of 100 mg/ml PDLAG in MS on nickel slides IX with an SMP4 pin and print time of 50 ms

b. Let dry for 10 min

c. Change pins and print drug on top of foundation layer with and SMP 10 pin 1-5X times (depending on how much drug is desired) with a print time of 100 ms letting spots dry between succesive “X”s.

d. Print MS only on top of spot with an SMP4 pin using 50 ms print time

e. Let slides dry at RT for at least 20 min

3. Coat slides according to the fibronectin coating protocol (see Example 3)

4. Place slides in either a 10 cm round or square petri dish adding 8e6 cells in 25 mls or 6.5e6 cells in 15 mls for each respectively

5. Incubate at 37C in C02 for 14 hours

### Example 7

This exemplification provides an alternate osmotic pump method for generating a test compound microarray with a PDLAG matrix.

1. Making solutions

a. PDLAG is dissolved in methyl Salicylate (MS) at 100 mg/ml

b. Drugs are dissolved at final concentration in 50:50 DMSO:water 0.2% type B gelatin solution.

c. Do NOT mix solutions prior to printing.

2. Printing:

constant factors throughout:

| | |
|---|---|
| preprint number | 20-40 |
| pre print time | 200 ms |
| drying time | 10 min |
| printing temp: | 27 C. |
| humidity: | 55% |
| slide type: | Nickel chelated from Xenopore |

a. Using the SMP10 pin print gelatin/drug solutions 1-5X times (depending on how much drug is desired) with a print time of 100 ms letting spots dry for 5 s between succesive “X”s.

b. Print 100 mg/ml PDLAG on top of spot with an SMP4 pin using 50 ms print time.

c. Let slides dry at RT for at least 20 min.

d. Prick the top of spot to penetrate the polymer creating a hole to the gelatin layer.

3. Coat slides according to the fibronectin coating protocol

4. Place slides in either a 10 cm round or square petri dish adding 8e6 cells in 25 mls or 6.5e6 cells in 15 mls for each respectively.

5. Incubate at 37C in CO2 for 14 hours.

### Example 8

This exemplification describes the printing of a test compound array containing 21 different test compounds, and the use of the array for testing effects of test compounds on cell viability and morphology.

Array spots were printed according to the basic procedure described in Example 6, briefly: an initial layer of 100 mg/ml PDLAG in MS was printed with an SMP4 pin on slides just after heating the polymer solution. After drying, an SMP10 pin was used to make spots of drug dissolved in DMSO onto the foundation spots (with drying in-between each successive spotting). Finally, MS only was printed IX over the existing spots. Once printed, all slides were coated with fibronectin for Ihour (all printing was done at least 20 min before fibronectin coating) (see Example 3). The slides was then incubated at 37C for 32 hours with 4e6 new A549cells. Control spots (no test compound) were also printed for comparison.

The test compounds used were: 2-Amino-6-purinethiol, 9-Aminoacridine Free Base, Actinomycin D, Camptothecin, Cantharidin, Crystal Violet, Digoxin, dihydroouabain, Echinomycin, emetine dihydrochloride crystalline, mitoxantrone, Paclitaxil, phenyl arsine oxide, Podophyllotoxin, Proscillaridin A, Sangivamycin, Streptonigrin, Valinomycin, Verrucarin A, Vinblastine Sulfate salt, Vinpocetine.

Each test compound was tested at three different concentrations, and effects on cell morphology and viability were assessed by one or two blinded observers. Many of the compounds showed concentration-dependent effects on cell viability and morphology. For example, phenyl arsine oxide caused a ring of clearing in the cells, with the size of the ring increasing with the phenyl arsine oxide concentration.

While this invention has been particularly shown and described with reference to particular embodiments thereof, it will be understood by those skilled in the art that various changes in form and details may be made therein without departing from the scope of the invention encompassed by the appended claims.

What is claimed is:

1. A small molecule array which comprises a surface having affixed thereto or therein, in discrete, defined locations, one or more small molecules to be assessed for their effect(s) on one or more observable properties of a biological sample, wherein the small molecules are affixed to the surface by means of a polymer that immobilizes the small molecules to the surface, permits release of the small molecules, permits attachment of cells plated thereon or therein and is not toxic to cells plated thereon.

2. The small molecule array of claim 1 that is a microarray, wherein the biological sample is cells and the polymer is a hydrogel or a biodegradable polymer.

3. The microarray of claim 2, wherein the surface is a slide.

4. The microarray of claim 2, wherein the hydrogel is selected from the group consisting of: a methacrylate-based polymer, a polycarboxylic acid, a cellulosic polymer, polyvinylpyrrolidone, maleic anhydride polymer, polyamide, polyvinyl alcohol and polyethylene oxide.

5. The microarray of claim 2, wherein the biodegradable polymer is selected from the group consisting of: gelatin, poly (lactic acid), poly (glycolic acid) and poly lactide coglycolide.

6. The microarray of claim 2 that comprises from about 1 discrete, defined location per cm² to about 1,000,000 discrete, defined locations per cm².

7. A small molecule microarray that comprises a slide having affixed thereto, in discrete, defined locations, small molecule-containing hydrogel spots.

8. The small molecule microarray of claim 7, wherein the hydrogel spots are methacrylate-based hydrogel disks and the microarray comprises from about 1 spot per cm² to about 1,000,000 disks per cm².

9. The small molecule microarray of claim 8 which comprises from about 10 spots per cm² to about 1,000,000 disks per cm².

10. A small molecule microarray that comprises a slide having affixed thereto or therein, in discrete, defined locations, small molecule-containing biodegradable polymer spots.

11. The small molecule microarray of claim 10, wherein the microarray comprises from about 1 spot per cm² to about 1,000,000 spots per cm².

12. The small molecule microarray of claim 11, wherein the microarray comprises from about 10 spots per cm² to about 1,000,000 spots per cm².

13. The microarray of claim 1, wherein the polymer erodes in the presence of the cells plated thereon or therein, thereby releasing a portion of the small molecules.

14. The microarray of claim 1, wherein the polymer erodes at a measurable rate of erosion and the small molecules are released at a measurable rate of release, and wherein the rate of release is roughly proportional to the rate of erosion.

15. The microarray of claim 1, wherein the polymer adheres to the surface at the discrete, defined locations such that, after a 24 hour exposure to a cell culture medium, at least 95% of the discrete, defined locations have polymer adherent thereto or therein.

16. A small molecule-cell array which comprises: (a) a small molecule to be assessed for its effect on a phenotypic characteristic of cells and (b) cells on which an effect of the small molecule is to be assessed, wherein the small molecule is affixed to or within the surface of the array in discrete, defined locations by means of a polymer that immobilizes the small molecule to or within the surface, permits release of the small molecule and attachment of cells plated thereon or therein and is not toxic to cells plated thereon or therein and the cells are plated thereon or therein.

17. The small molecule-cell array of claim 16 that is a small molecule-cell microarray, wherein the surface is a slide and the polymer is a hydrogel or a biodegradable polymer.

18. The small molecule-cell microarray of claim 17, wherein the hydrogel is selected from the group consisting of: a methacrylate-based polymer, a polycarboxylic acid, a cellulosic polymer, polyvinylpyrrolidone, maleic anhydride polymer, polyamide, polyvinyl alcohol and polyethylene oxide.

19. The small molecule-cell microarray of claim 17, wherein the biodegradable polymer is selected from the group consisting of: gelatin, poly (lactic acid), poly (glycolic acid) and poly lactide coglycolide.

20. The small molecule-cell microarray of claim 19 that comprises from about 1 discrete, defined location per cm² to about 1,000,000 discrete, defined locations per cm².

21. The small molecule-cell microarray of claim 20 that comprises from about 10 discrete, defined locations per cm² to about 1,000,000 discrete, defined locations per cm².

22. A small molecule-cell microarray that comprises a surface having affixed thereto or therein, in discrete, defined locations, test small molecule-containing hydrogel spots and cells plated thereon or therein.

23. The small molecule-cell microarray of claim 22, wherein the hydrogel spots are methacrylate-based hydrogel spots and the microarray comprises from about 1 spot per cm² to about 1,000,000 spots per cm².

24. A small molecule-cell microarray that comprises a surface having affixed thereto or therein, in discrete, defined locations, test small molecule-containing biodegradable polymer spots and cells plated thereon or therein.

25. The small molecule-cell microarray of claim 24, wherein the microarray comprises from about 1 spot per cm² to about 1,000,000 spots per cm².

26. The small molecule-cell microarray of claim 25, wherein the microarray comprises from about 10 spots per cm² to about 1,000,000 spots per cm².

27. A method of producing a small molecule array, wherein the small molecule array comprises test small molecule-containing hydrogel spots arrayed on a surface, comprising: (a) arraying a test small molecule-hydrogel solution on or within a surface in discrete, defined locations, thereby producing a surface bearing or containing the test small molecule-hydrogel solution in discrete, defined locations and (b) subjecting the surface produced in (a) to conditions under which polymerization of the solution occurs, whereby the solution is polymerized and affixed to or within the surface as test small molecule-containing hydrogel spots, thereby producing the small molecule array.

28. The method of claim 27, wherein the surface is a slide and the hydrogel is a methacrylate-based hydrogel.

29. The method of claim 28, wherein the small molecule array is a small molecule microarray.

30. A method of producing a small molecule array, wherein the small molecule array comprises test small molecule-containing biodegradable spots arrayed on a surface, comprising: (a) arraying a test small molecule-biodegradable polymer solution on or in a surface in discrete, defined locations, thereby producing a surface bearing the test small molecule-biodegradable polymer solution in discrete, defined locations and (b) subjecting the surface produced in (a) to conditions under which polymerization of the solution occurs, whereby the solution is polymerized and affixed to or in the surface as test small molecule-containing biodegradable polymer spots, thereby producing the small molecule array.

31. The method of claim 30, wherein the surface is a slide.

32. The method of claim 31, wherein the small molecule array is a small molecule mi cro array.

33. A method of identifying a small molecule that has an effect on a phenotypic characteristic of cells, comprising plating the cells on or in a small molecule array, wherein the small molecule array comprises a surface having affixed thereto or therein, in discrete, defined locations, test small molecule-containing hydrogel spots, thereby producing a small molecule-cell array; maintaining the small molecule-cell array under conditions under which small molecule is released from the hydrogel spots and contacts membranes of vicinal cells;
observing effects of the small molecule on phenotypic characteristics of the vicinal cells, wherein if a phenotypic characteristic of vicinal cells is altered, the test small molecule is a small molecule that has an effect on a phenotypic characteristic of the cells.

34. The method of claim 33, wherein the small molecule array is a small molecule microarray and the surface is a slide.

35. The method of claim 34, wherein the hydrogel is a methacrylate-based hydrogel.

36. A method of identifying a small molecule that has an effect on a phenotypic characteristic of cells, comprising plating the cells on or in a small molecule array, wherein the small molecule array comprises a surface having affixed thereto or therein, in discrete, defined locations, test small molecule-containing biodegradable polymer spots, thereby producing a small molecule-cell array;
maintaining the small molecule-cell array under conditions under which small molecule is released from the biodegradable polymer spots and contacts membranes of vicinal cells; observing effects of the small molecule on phenotypic characteristics of the vicinal cells, wherein if a phenotypic characteristic of vicinal cells is altered, the test small molecule is a small molecule that has an effect on a 5 phenotypic characteristic of the cells.

37. The method of claim 36, wherein the small molecule array is a small molecule microarray and the surface is a slide.

38. The method of claim 37, wherein the small molecule microarray comprises from about 1 discrete, defined location per cm² to about 1,000,000 discrete, defined locations per cm².

39. The method of claim 38, wherein the small molecule microarray comprises from about 10 discrete, defined locations per cm² to about 1,000,000 discrete, defined locations per cm² .
